(19) United States
(12) Patent Application Publication
(10) Pub. No.: US 2008/0064679 A1
(43) Pub. Date: Mar. 13, 2008

(54) Water Soluble Cannabinoids

(76) Inventors: Billy R. Martin, Richmond, VA (US); Raj K. Razdan, Gloucester, MA (US); Anu Mahadevan, Westford, MA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/571,059

(22) PCT Filed: Jun. 23, 2005
(86) PCT No.: PCT/US05/22178

§ 371 Date: Nov. 21, 2007

Related U.S. Application Data
(60) Provisional application No. 60/582,076, filed on Jun. 24, 2004.

(51) Int. Cl. A61K 31/5377; A61K 31/352; A61K 31/4025; A61K 31/422; A61K 31/55; C07D 311/80; C07D 413/02; C07D 417/02; C07D 413/14; C07D 405/02; A61P 43/00; A61K 31/427; A61K 31/453; A61K 31/496

(52) U.S. Cl. 514/212.01; 514/232.5; 514/232.8; 514/254.11; 514/320; 514/365; 514/374; 514/422; 514/454; 540/596; 544/111; 544/150; 544/375; 546/196; 548/200; 548/235; 548/525; 549/390

(57) ABSTRACT

Water-soluble cannabinoid compounds that are agonists of CB₁ and CB₂ cannabinoid receptors are provided. The compounds are made water-soluble by derivatization of the alkyl side chain and/or the phenolic hydroxyl group of tetrahydrocannabinol. The water-soluble cannabinoids are useful for the treatment of appetite loss, pain, multiple sclerosis, nausea and vomiting, and epilepsy.

### BACKGROUND OF THE INVENTION

1. Field of the Invention The invention generally relates to water-soluble cannabinoid agonists. In particular, the invention provides water-soluble cannabinoid compounds that are agonists of CB₁ and CB₂ receptors, and that are useful for treating or alleviating a number of disorders of symptoms thereof, including, for example, appetite loss, pain, multiple sclerosis, nausea and vomiting, and epilepsy.

2. Background of the Invention

Marijuana has attracted considerable attention for centuries because of its psychotropic and medicinal properties. Early scientific investigations were conducted with either smoked plant material or the plant extract. Needless to say, the synthesis of marijuana's major psychotropic constituent, Δ⁹-THC, opened a new era in marijuana research (Gaoni and Mechoulam, 1964). For the first time researchers were able to conduct research in a quantitative fashion, because the precise dose of Δ⁹-THC could be administered. Unfortunately, Δ⁹-THC is a non-crystalline, highly lipophilic compound that requires solubilization with either a surfactant agent or adherence to a water miscible substance (albumin, Tween 80, etc.). Even under these circumstances, Δ⁹-THC is sometimes capable of adhering to solid surfaces rather than remaining in solution. This high lipophilicity has placed constraints on the pharmacological evaluation of Δ⁹-THC. There is always the concern that the use of different vehicles in separate pharmacological studies may influence the pharmacological effects of Δ⁹-THC. It is for these reasons that there have been numerous attempts to prepare water-soluble derivatives of cannabinoids.

The first successful attempt in preparing a water-soluble form of Δ⁹-THC involved converting it to a morpholinobutyrl ester, the hydrochloride of which was water-soluble (Zitko et al., 1972). This compound retained cannabinoid pharmacological activity. A morpholinobutyrl ester of Δ⁸-THC was also found to be equipotent to Δ⁸-THC in several behavioral models (Compton and Martin, 1990). Water-soluble derivatives of THC were prepared in other laboratories and found to be effective in lowering intraocular pressure in rabbits (ElSohly et al., 1984). In more recent times, numerous cannabinoid analogs have been developed that are considerably more potent than Δ⁹-THC (Martin et al., 1999; Khanolkar et al., 2000). One of these compounds contains a cyano group on the terminal carbon atom of the side chain in Δ⁸-THC (Martin et al., 1999). Therefore, a morpholinobutryl ester of this potent cannabinoid was prepared and found to be highly active when prepared in saline and evaluated either in vivo or in vitro (Pertwee et al., 2000). It is assumed that these phenolic esters (Zitko et al., 1972; Pertwee et al., 2000) are prodrugs, because a free hydroxyl group is required for pharmacological activity of Δ⁹-THC at the CB₁ cannabinoid receptor (Razdan, 1986; Huffman et al., 2002). Phosphate esters of the endocannabinoids anandamide and noladin ether have also been prepared (Juntunen et al., 2003a; Juntunen et al., 2003b). These esters are rapidly hydrolyzed in biological tissues and are effective in lowering intraocular pressure in rabbits when applied in an aqueous solution.

There have also been numerous attempts to prepare analogs with reduced lipophilicity. Early receptor binding studies conducted with radiolabeled Δ⁸-THC were of limited success because of the extensive non-specific binding by this highly lipophilic agent (Harris et al., 1978). In an effort to reduce non-specific binding, Nye et al. (Nye et al., 1988) prepared a radiolabeled trimethylammonium analog of Δ⁸-THC. This charged analog allowed them to label a specific binding site in brain, although it remains to be established that this site is a true cannabinoid receptor. A nitrogen mustard analog of Δ⁹-THC was found to be behaviorally active when administered centrally but not when administered peripherally, possibly due to reduced lipophilicity (Little et al., 1987).

There is thus an ongoing need to provide water-soluble cannabinoid compounds exhibiting high CB₁ and CB₂ receptor affinity and high bioavailability.

### SUMMARY OF THE INVENTION

The invention provides water-soluble cannabinoid compounds with high CB₁ and CB₂ receptor affinity and high bioavailability. The compounds result from structural alterations in tetrahydrocannabinol for the purpose of increasing its water solubility and/or miscibility. By making structural alterations in the alkyl side chains and at the phenolic hydroxyl group of tetrahydrocannabinol, a series of analogs have been prepared that are soluble and/or miscible in water, and which show high bioavailability. The analogs exhibit high affinity for the CB₁ and CB₂ receptors, and are thus water-soluble cannabinoid agonists. The compounds are useful for treating diseases and disorders related to CB₁ and CB₂ receptor function, including appetite loss, nausea and vomiting, pain, multiple sclerosis and epilepsy. The agents are also valuable as research tools for scientists. In addition, novel analogs that are not water soluble but that exhibit high levels of CB₁ and CB₂ receptor affinity and in vivo activity are provided.

It is an object of this invention to provide water-soluble cannabinoid analogs with the general structure

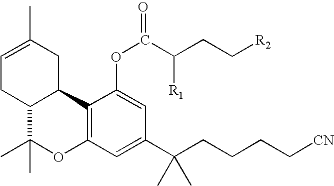

wherein R₁ is H or a straight-chained, branched or cyclic C₁-C₆ lower alkyl; and R₂ is a 5-7 membered heterocyclic ring in which at least one of the member atoms is N; or wherein R₂ is NR₃ where R₃ is H₂, H₃⁺, or mono or dialkyl C₁-C₆. Salts of such compounds are also contemplated. In one embodiment, the 5-7 membered heterocyclic ring may be, for example, piperidine, methyl piperidine, methyl piperazine or morpholine.

The invention also provides cannabinoid analogs with the general structure

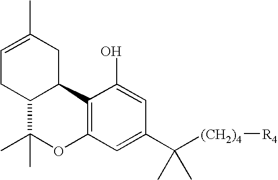

in which R₄ is an azole or morpholine ring. Salts of these compounds are also provided. In one embodiment, the azole ring may be, for example, imidazole, 1H-imidazole, methyl imidazole, pyrazole, and triazole. In another embodiment, the cannabinoid analog is a water-soluble salt and the azole ring may be, for example, imidazole 1H-imidazole, methyl imidazole.

The invention also provides cannabinoid analogs with the general structure

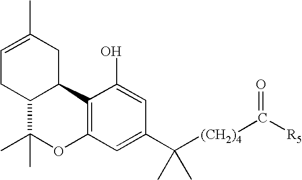

in which R₅ is NH₂, NHCH₃, or NHR₆, and R₆ is a 5-7 membered heterocyclic ring in which at least one of the member atoms is N. Alternatively, R₅ may be a 5-7 membered heterocyclic ring in which at least one of the member atoms is N. Salts of such compounds are also provided. In a preferred embodiment of the invention, R₆ is a heterocyclic ring such as, for example, morpholine, homo-piperidine, pyrrolidine, or piperidine. In another embodiment, R₅ is a heterocyclic ring such as, for example, morpholine, piperidine, piperizine, pyrrolidine, or homo-piperidine.

The invention also provides water-soluble cannabinoid analogs with general structure

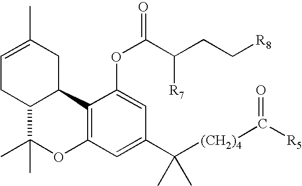

in which R₇ is H or a straight-chained, branched or cyclic C₁-C₆ lower alkyl (e.g. CH₃); and R₈ is a 5-7 membered heterocyclic ring in which at least one of the member atoms is N; or NR₃ where R₃ is H₂, H₃⁺, or mono or dialkyl C₁-C₆; and wherein R₉ is NH₂, NHCH₃,or NHR₆, where R₆ is a 5-7 membered heterocyclic ring in which at least one of the member atoms is N. Alternatively, R₉ may be a 5-7 membered heterocyclic ring in which at least one of the member atoms is N. Salts of such compounds are also contemplated. In a preferred embodiment of the invention, the water-soluble cannabinoid is

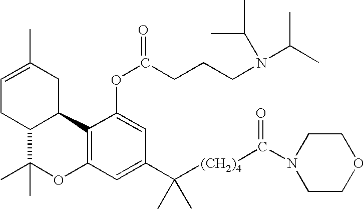

In addition, a cannabinoid analog with the general structure

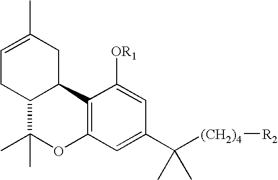

is provided. In this embodiment of the invention, R₁═H, —COCHR₃—CH₂—CH₂-R₄ and R₂═CN or COR₇, where: R₃═H, straight-chained, branched or cyclic C₁-C₆ lower alkyl; R₄ and R₇ may be the same or different and are: NH₂, NHCH₃, N(R₈)₂ (where R₈=COR₇); a 5-7 membered heterocylic ring with at least one N atom; or NHR₅ (where R₅ is a 5-7 membered heterocyclic ring with one N atom). In a preferred embodiment, the 5-7 membered heterocyclic ring is

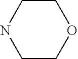

The invention also provides a method of treating or alleviating symptoms of a disease or disorder associated with CB1 and CB2 cannabinoid receptors in a patient in need thereof. The method comprises the step of administering to the patient a compound (or salt thereof) of a general structural formula:

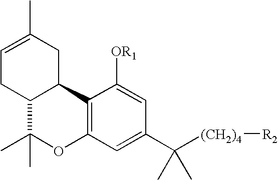

in which R₁═H, —COCHR₃—CH₂—CH₂-R₄ and R₂═CN or COR₇, where: R₃═H, straight-chained, branched or cyclic C₁-C₆ lower alkyl; R₄ and R₇ may be the same or different and are: NH₂, NHCH₃, N(R₈)₂ (where R₈═COR₇); a 5-7 membered heterocylic ring with at least one N atom; or NHR₅ (where R₅ is a 5-7 membered heterocyclic ring with one N atom). In a preferred embodiment, the 5-7 membered heterocyclic ring is

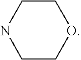

### BRIEF DESCRIPTION OF THE DRAWINGS

FIG. 1. General synthesis scheme for compounds of the present invention.

FIG. 2. Synthesis Scheme 1 for selected compounds shown in Table 3 (e.g. 16a-e, 17a-e).

FIG. 3. Synthesis Scheme 2 for selected compounds shown in Table 1 (e.g. 7a-g).

FIG. 4. Synthesis Scheme 3 for selected compounds shown in Table 1 (e.g. 5c-f).

FIG. 5. Synthesis Scheme 4 for selected compounds shown in Table 2 (e.g. 5h, 5j-m, 7r).

FIG. 6. Synthesis Scheme 5 for 7m, shown in Table 4.

FIG. 7. Effects of O-2426 (0.1 mg/kg) or saline administered i.v. at different time points before testing. The ED50 of O-2426 after i.v. administration is 0.05, 0.04, 0.10 and 0.11 μmoles/kg in spontaneous activity, tail-flick response, rectal temperature and relative immobility, respectively. Therefore a time course was determined using a dose of 0.1 mg/kg (0.16 μmoles/kg). The data in the figure show that the sedative effects of this ester have disappeared by one hour but the other effects remain. All effects are gone by 2 hours.

FIG. 8. Time course of O-2486 (1 mg/kg) or saline after i.v. administration at different time points before testing. The ED50's of O-2486 after i.v. administration are 0.05, 0.20, 1.7, and 0.6 μmoles/kg in spontaneous activity, tail-flick, rectal temperature and relative immobility, respectively. Therefore, a dose of 1 mg/kg (1.6 μmoles/kg) was chosen to determine the time course. The analgesic and relative immobility effects of O-2486 were still present at two hours but not sedation and hypothermia. It was anticipated this analog would have a somewhat longer duration of action because of the alpha methyl substitution which theoretically should retard hydrolysis. Indeed, this analog has a longer duration of action than O-2426. However, direct comparison is complicated by the fact that a much higher dose of O-2486 was required because it is less potent than O-2426. It does appear that there is some separation of pharmacological actions with O-2486 and not O-2426.

FIG. 9. Time course of O-2485 or saline after i.v. administration. The ED50's of O-2485 after i.v. administration were 0.45, 1.0, 1.9 and 1.9 μmoles/kg in spontaneous activity, tail-flick, rectal temperature and relative immobility, respectively. The time course was carried out with a dose of 3 mg/kg (5 μmoles/kg). It is evident that the antinociceptive effect of this analog is retained even after recovery from sedation. At four hours, antinociception is present when almost all other effects have dissipated.

FIG. 10. Time course of O-2545 or saline after i.v. administration. The ED50's of O-2545 were 0.09, 0.16, 0.29 and 0.14 μmoles/kg. Therefore, a dose of 0.24 μmoles/kg, i.v., was used for the time course. At 1 hr all effects, except ring immobility, are present but only antinociception is present at 2 hours.

FIG. 11. Time course of O-2716 or vehicle (1:1:18 ethanol:emulphor:saline) after i.v. administration. The ED50's of O-2716 after i.v. administration were 0.3, 0.9, 0.15 and 0.20 μmoles/kg for spontaneous activity, tail-flick, rectal temperature and relative immobility, respectively. Therefore, a dose of 0.1 mg/kg (0.24 μmoles/kg) was used for the time course. This analog was relatively short acting with most of the effects gone by 1 hour and no separation of effects.

FIG. 12. Time course of O-2715 or vehicle (1:1:18 ethanol:emulphor:saline) after i.v. administration. The ED50's of O-2715 after i.v. administration were 0.004, 0.06, 0.14 and 0.07 μmoles/kg in spontaneous activity, tail-flick, rectal temperature, and relative immobility, respectively. A dose of 0.3 mg/kg (0.74 μmoles/kg) was chosen for the time course. The duration of antinociception and hypothermia exceeded that of sedation and relative immobility.

FIG. 13. Potency of O-2426 following oral administration 30 minutes before the start of testing. The results demonstrate that this analog is effective in all four tests at a dose of 1 mg/kg.

FIG. 14. Potency of O-2486 after oral administration 30 minutes before the start of testing. The results show that a dose of 10 mg/kg is highly effective in all four pharmacological measures.

FIG. 15. Potency of O-2485 after oral administration 30 minutes before the start of testing. This analog is active in all measures following an oral dose of 10 mg/kg.

FIG. 16. Potency of O-2545 following oral administration 30 minutes before the start of testing. A dose of 10 mg/kg was fully effective in producing antinociception, hypothermia and ring immobility but was only a partial agonist in producing sedation.

FIG. 17. Time course of O-2545 or vehicle (1:1:18 ethanol:emulphor:saline) following oral administration at different time points before testing. A dose of 10 mg/kg was used and was found to produce the full range of effects up to 2 hours.

FIG. 18. Potency of O-2715 following oral administration 30 minutes before the start of testing. A dose of 10 mg/kg was active in all tests.

FIG. 19. Time course of O-2715 (10 mg/kg) or vehicle (1:1:18 ethanol:emulphor:saline) following oral administration at different time points before testing. This dose produced effects that lasted only 1 hour.

FIG. 20. Potency of O-2716 following oral administration 30 minutes before the start of testing. The maximum dose tested, 10 mg/kg, failed to produce complete antinociception.

FIG. 21. Time course of O-2716 (10 mg.kg) or vehicle (1:1:18 ethanol:emulphor:saline) following oral administration at different time points before testing. The effects lasted at least 2 hours.

### DETAILED DESCRIPTION OF THE PREFERRED EMBODIMENTS OF THE INVENTION

The invention provides water-soluble cannabinoid analogs with high CB₁ and CB₂ receptor affinity and high bioavailability. Production of the compounds was based on two approaches to modification of tetrahydrocannabinol: structural alterations in 1) the alkyl side chains; and 2) at the phenolic hydroxyl group. The resulting series of analogs are soluble and/or miscible in water, show high bioavailability, and exhibit high affinity for the CB₁ and CB₂ receptors (i.e. they are cannabinoid agonists.)

By “water-soluble” we mean that 1 mg of material in 1 ml of water gives a clear solution and is water miscible.

By “high affinity” we mean that the compounds exhibit a Ki in the range of about 0.03 nM to about 80 nM, and preferably from about 0.03 nM to about 50 nM, for either the CB₁ or CB₂ receptors, or both.

In one embodiment of the invention, the structure of the water-soluble cannabinoid analog is as depicted in Formula 1:

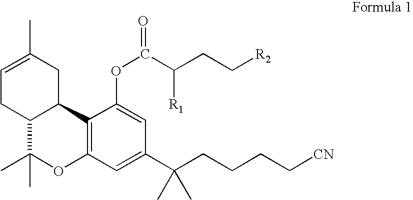

In this structure R₁ may be H, or a C1-C6 lower alkyl group and may be straight-chained, branched or cyclic (e.g. CH₃). R₂ may be a 5-7 membered heterocyclic ring in which at least one of the member atoms is N. Examples of suitable 5-7 membered heterocyclic rings include but are not limited to piperidine, methyl piperidine, methyl piperazine, morpholine, etc. Alternatively, R₂ may be NR₃ where R₃ is H₂, H₃⁺, or mono or dialkyl C₁-C₆ (e.g. (CH₃)₂, CH(CH₃)₂, C(CH₃)₃, etc).

The invention further comprehends salts of the compounds depicted in Formula 1. Examples of suitable types of salts include but are not limited to HCl, iodine, ammonia, sulfates, tartrates, succinates, quaternary salts, etc. Those of skill in the art will recognize that any salts of the compounds may be used, so long as the salt retains water solubility.

In another aspect of the invention, a cannabinoid analog with a structure as presented in structural Formula 2 is provided.

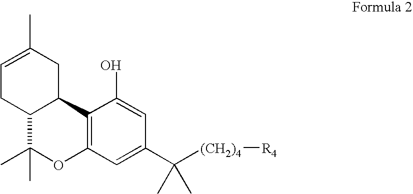

### In compounds of Formula 2, R₄ is a heterocyclic ring such as an azole or morpholine ring, examples of which include but are not limited to imidazole, 1H-imidazole, methyl imidazole, pyrazole, triazole, and the like.

In addition, the compound may be provided as a salt (e.g. HCl, iodine, ammonia, sulfates, tartrates, succinates, quaternary salts, etc.). Those of skill in the art will recognize that any salts of the compounds may be used, so long as the salt retains water solubility.

In a preferred embodiments, the cannabinoid analog of Formula 2 is a water-soluble salt, and the heterocyclic ring is an imidazole ring (such as 1H-imidazole, methyl imidazole, etc.) or a morpholine ring.

In yet another aspect of the invention, cannabinoid analogs with the structure depicted in Formula 3 (and their salts, e.g. HCl, iodine, ammonia, sulfates, tartrates, succinates, quaternary salts, etc.) are provided. Those of skill in the art will recognize that any salts of the compounds may be used, so long as the salt retains water solubility.

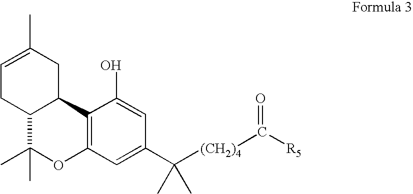

In compounds of Formula 3, R₅ may be, for example, NH₂, NHCH₃, or NHR₆, where R₆ is a 5-7 membered heterocyclic ring in which at least one of the member atoms is N. Suitable examples of such heterocyclic rings include but are not limited to morpholine, homo-piperidine, pyrrolidine, piperidine, etc. Alternatively, R₅ may be a 5-7 membered heterocyclic ring in which at least one of the member atoms is N, suitable examples of which include but are not limited to morpholine, piperidine, piperizine, pyrrolidine, homo-piperidine, etc.

In yet another embodiment, the invention provides water-soluble cannabinoid analogs with the structure depicted in Formula 4 and their water soluble salts (e.g. HCl, iodine, ammonia, sulfates, tartrates, succinates, quaternary salts, etc.).

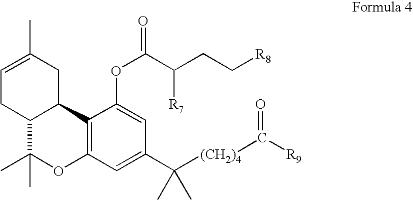

In Formula 4, R₇ may be H, or a C1-C6 lower alkyl group and may be straight-chained, branched or cyclic (e.g. CH₃). R₈ may be a 5-7 membered heterocyclic ring in which at least one of the member atoms is N. Examples of suitable 5-7 membered heterocyclic rings include but are not limited to piperidine, methyl piperidine, methyl piperazine, morpholine, etc. Alternatively, R₈ may be NR₃ where R₃ is H₂, H₃⁺, or mono or dialkyl C₁-C₆ (e.g. (CH₃)₂, CH(CH₃)₂, C(CH₃)₃, etc).

In compounds of Formula 4, R₉ may be, for example, NH₂, NHCH₃, or NHR₆, where R₆ is a 5-7 membered heterocyclic ring in which at least one of the member atoms is N. Suitable examples of such heterocyclic rings include but are not limited to morpholine, homo-piperidine, pyrrolidine, piperidine, etc. Alternatively, R₉ may be a 5-7 membered heterocyclic ring in which at least one of the member atoms is N, suitable examples of which include but are not limited to morpholine, piperidine, piperizine, pyrrolidine, homo-piperidine, etc.

In one embodiment of the invention, the compound of Formula 4 is as depicted in Formula 5.

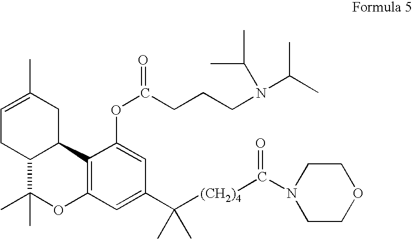

### In addition, the invention provides a cannabinoid analog with general Formula 6

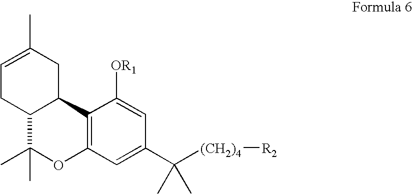

In compounds of Formula 6, R₁═H, —COCHR₃—CH₂—CH₂-R₄; R₂═CN or COR₇; R₃═H, straight-chained, branched or cyclic C₁-C₆ lower alkyl. In the formula, R₄ and R₇ may be the same or different and are: NH₂, NHCH₃, N(R₈)₂, where R₈═COR₇; or a 5-7 membered heterocylic ring with at least one N atom; or NHR₅, where R₅ is a 5-7 membered heterocyclic ring with one N atom. In one embodiment of the invention, the 5-7 membered heterocyclic ring is

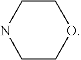

The compounds of the present invention are useful for a variety of therapeutic applications. For example, the compounds are useful for treating or alleviating symptoms of diseases and disorders involving CB₁ and CB₂ receptors, including appetite loss, nausea and vomiting, pain, multiple sclerosis and epilepsy. For example, they may be used to treat pain (i.e. as analgesics) in a variety of applications including but not limited to pain management. By “treating” we mean that the compound is administered in order to alleviate symptoms of the disease or disorder being treated. Those of skill in the art will recognize that the symptoms of the disease or disorder that is treated may be completely eliminated, or may simply be lessened. Further, the compounds may be administered in combination with other drugs or treatment modalities.

It is well documented that agents that activate CB₁ cannabinoid receptors stimulate appetite, nausea and vomiting, and pain (Martin B. R. and Wiley, J. L, Mechanism of action of cannabinoids: how it may lead to treatment of cachexia, emesis and pain, Journal of Supportive Oncology 2: 1-10, 2004), multiple sclerosis (Pertwee, R. G., Cannabinoids and multiple sclerosis, Pharmacol. Ther. 95, 165-174, 2002) and epilepsy (Wallace, M. J., Blair, R. E., Falenski, K. WW., Martin, B. R., and DeLorenzo, R. J. Journal Pharmacology and Experimental Therapeutics, 307: 129-137, 2003). In addition, CB₂ receptor agonists have been shown to be effective in treating pain (Clayton N., Marshall F. H., Bountra C., O'Shaughnessy C. T., 2002. CB1 and CB2 cannabinoid receptors are implicated in inflammatory pain. 96, 253-260; Malan T. P., Ibrahim M. M., Vanderah T. W., Makriyannis A., Porreca F., 2002. Inhibition of pain responses by activation of CB(2) cannabinoid receptors. Chemistry and Physics of Lipids 121, 191-200; Malan T. P., Jr., Ibrahim M. M., Deng H., Liu Q., Mata H. P., Vanderah T., Porreca F., Makriyannis A., 2001. CB2 cannabinoid receptor-mediated peripheral antinociception. 93, 239-245.; Quartilho A., Mata H. P., Ibrahim M. M., Vanderah T. W., Porreca F., Makriyannis A., Malan T. P., Jr., 2003. Inhibition of inflammatory hyperalgesia by activation of peripheral CB2 cannabinoid receptors. Anesthesiology 99, 955-960) and multiple sclerosis (Pertwee, R. G., Cannabinoids and multiple sclerosis, Pharmacol. Ther. 95, 165-174, 2002) in animal models. The compounds described herein have high affinity for both CB₁ and CB₂ receptors and produce cannabinoid in vivo effects. These compounds are also effective analgesics in the radiant heat model of pain as measured by the tail-flick response (see Examples).

Implementation will generally involve identifying patients suffering from the indicated disorders and administering the compounds of the present invention in an acceptable form by an appropriate route. The exact dosage to be administered may vary depending on the age, gender, weight and overall health status of the individual patient, as well as the precise etiology of the disease. However, in general for administration in mammals (e.g. humans), dosages in the range of from about 0.1 to about 30 mg of compound per kg of body weight per 24 hr., and more preferably about 0.1 to about 10 mg of compound per kg of body weight per 24 hr., are effective.

Administration may be oral or parenteral, including intravenously, intramuscularly, subcutaneously, intradermal injection, intraperitoneal injection, etc., or by other routes (e.g. transdermal, sublingual, oral, rectal and buccal delivery, inhalation of an aerosol, etc.). In a preferred embodiment of the invention, the water-soluble cannabinoid analogs are provided orally or intravenously.

In particular, the phenolic esters of the invention (Formula 1) are preferentially administered systemically in order to afford an opportunity for metabolic activation via in vivo cleavage of the ester. In addition, the water soluble compounds with azole moieties at the pentyl side chain (Formula 2, e.g. with imidazole moieties) do not require in vivo activation and may be suitable for direct administration (e.g. site specific injection).

The compounds may be administered in the pure form or in a pharmaceutically acceptable formulation including suitable elixirs, binders, and the like (generally referred to a “carriers”) or as pharmaceutically acceptable salts (e.g. alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil based vehicles. Water may be used as the carrier for the preparation of compositions (e.g. injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc). Solid diluents and excipients include lactose, starch, conventional disintergrating agents, coatings and the like. Preservatives such as methyl paraben or benzalkium chloride may also be used. Depending on the formulation, it is expected that the active composition will consist of about 1% to about 99% of the composition and the vehicular “carrier” will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, bolus dose, or at a gradual or continuous, constant or controlled rate to a patient. In addition, the time of day and the number of times per day that the pharmaceutical formulation is administered may vary are and best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities. The compounds may also be added to blood ex vivo and then be provided to the patient.

The cainabinoid analogs of the invention are also valuable as research tools, e.g. for investigational purposes.

### EXAMPLES

### Example 1

Nonstandard abbreviations: Δ⁹-THC, Δ⁹-tetrahydrocannabinol; % MPE, percent maximum possible effect; CB, cannabinoid receptor; CP55940, (−)-3-[2-hydroxy-4-(1,1-dimethylheptyl) phenyl]-4-(3-hydroxypropyl)cyclohexan-1-ol.

### Introduction

Presently, there are numerous structural classes of cannabinoid receptor agonists, all of which require solubilization for experimental purposes because of their water-insolubility. One strategy for solubilizing water-soluble tetrahydrocannabinols is conversion of the phenolic hydroxyl to a morpholinobutyryloxy substituent. The hydrochloride salts of these analogs are water-soluble and active in vivo when administered in saline. The present investigation demonstrates that an array of hydrochloride salts of substituted butyryloxy esters are water-soluble and highly potent. The substitutions include piperidine, piperazine, and alkyl substituted amino moieties. It was also discovered that incorporation of a nitrogenous moiety in the alkyl side chain of tetrahydrocannabinol increased pharmacological potency. A series of carboxamido analogs exhibited high pharmacological potency but their hydrochloride salts were not water-soluble. On the other hand, incorporation of imidazoles in the terminus of the side chain led to water-soluble hydrochloride salts that were highly potent when administered in saline to laboratory animals. It is now possible to conduct cannabinoid research with agonists that are water-soluble and thus obviating the need of solubilizing agents.

An objective of the investigations noted below was to explore possible structural alterations in the THC structure that would render it water-soluble. An additional objective was to develop a series of water-soluble analogs that were not prodrugs.

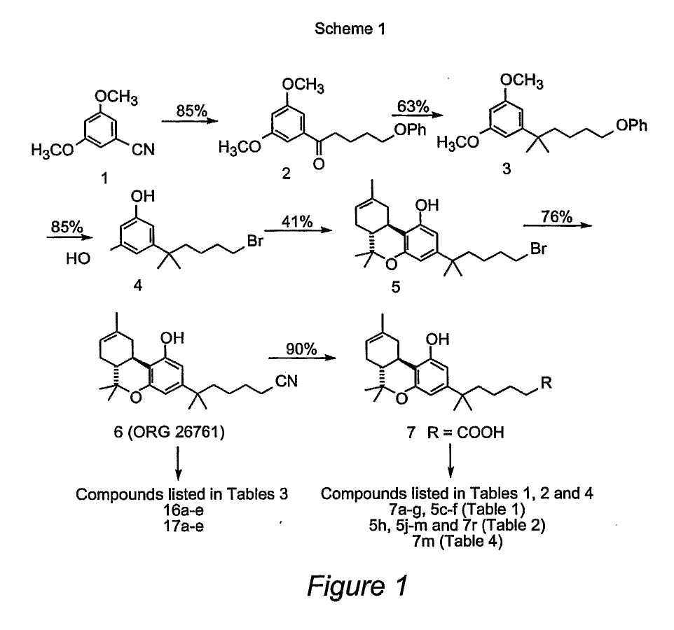

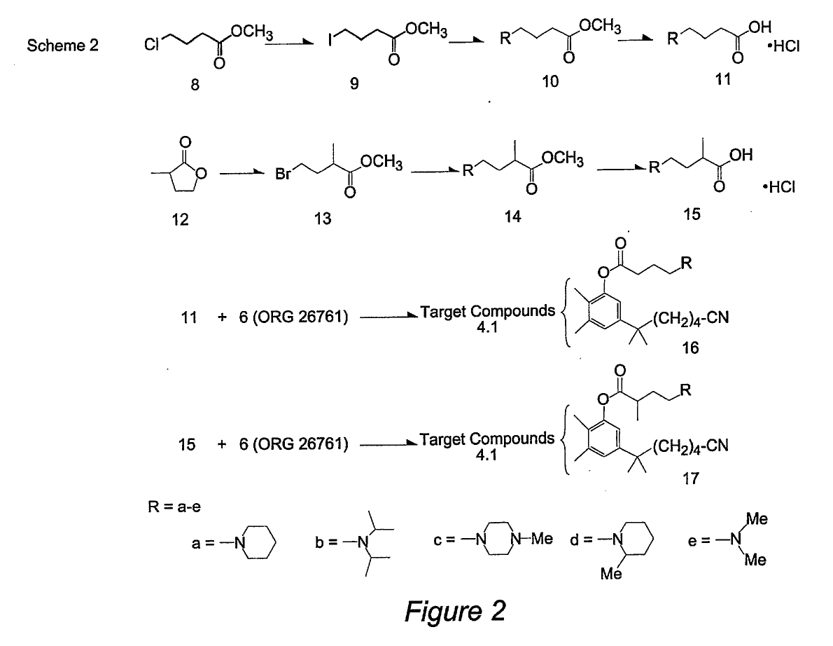

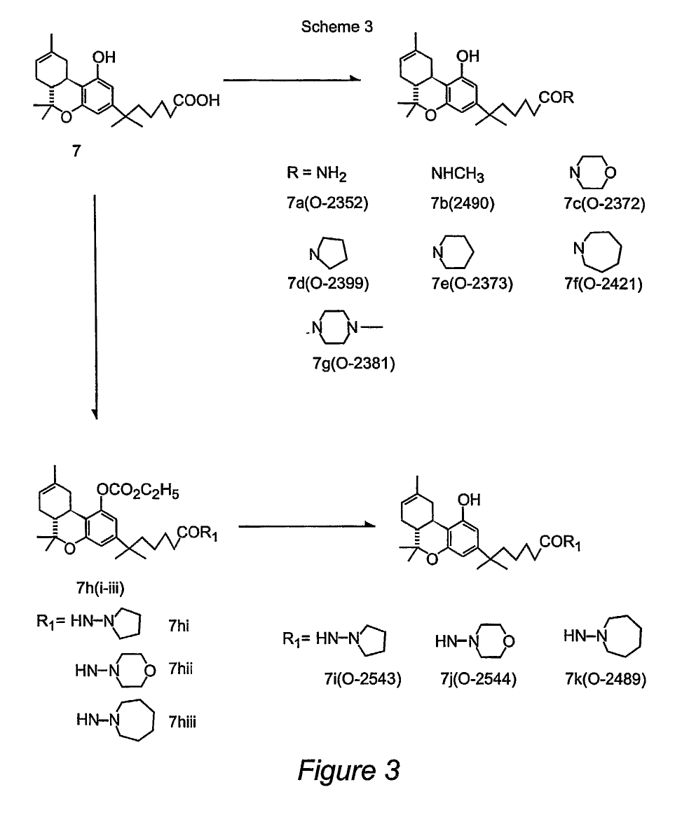

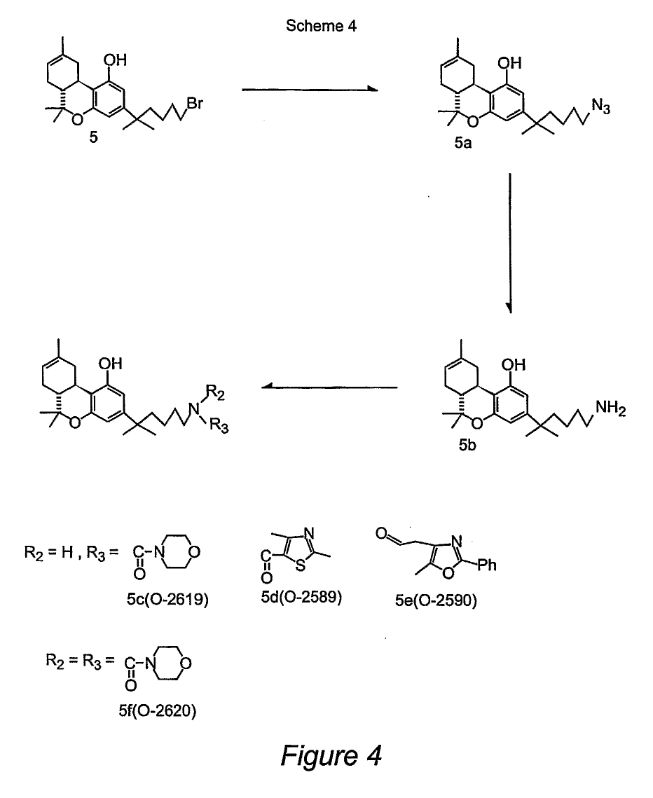

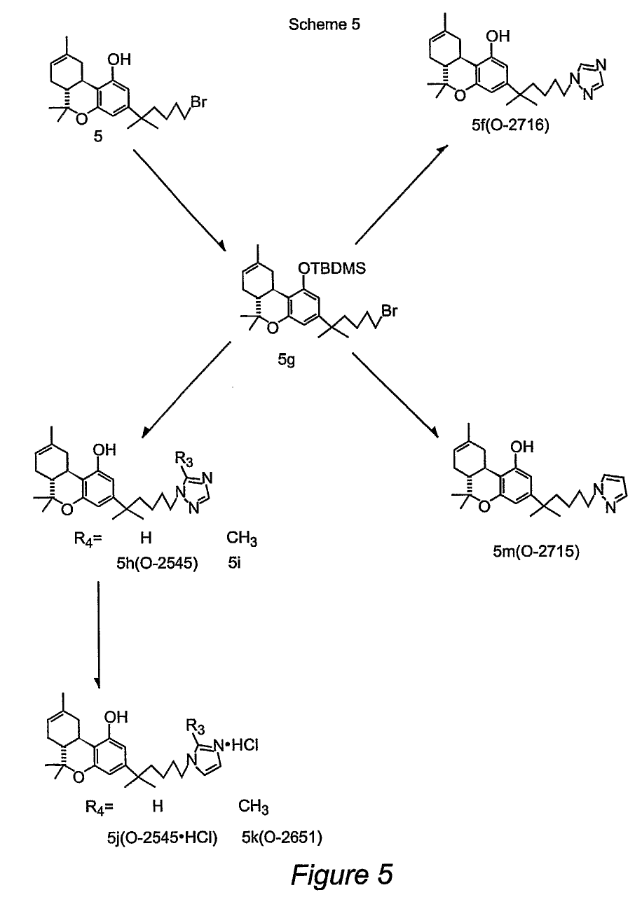

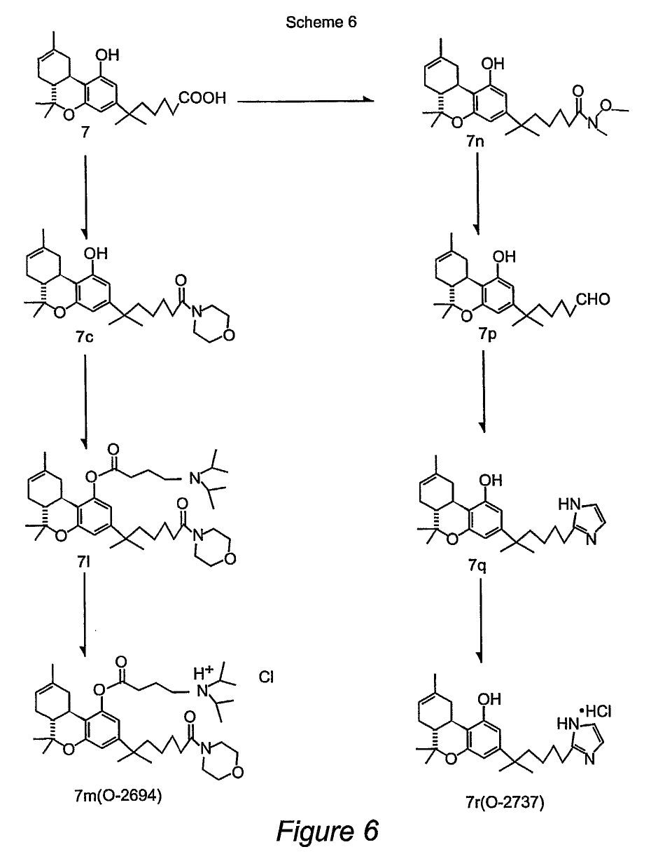

Materials and Methods. Male ICR mice (Harlan Laboratories, Indianapolis, Ind.) weighing between 24 to 30 g were used in all experiments. Mice were maintained on a 14: 10-hr light/dark cycle with food and water available ad lib. All test groups consisted of 6 to 12 mice. THC was obtained from NIDA and dissolved in a vehicle consisting of ethanol, emulphor and saline in a ratio of 1:1:18. Analogs were dissolved either in the vehicle or saline depending upon their water solubility. All chemicals were purchased from Sigma (St. Louis, Mo.) except the following: [³⁵S]GTPγS (1250 Ci/mmol) was purchased from New England Nuclear Group (Boston, Mass.), GTPγS from Boehringer Mannheim (New York, N.Y.), Dulbeco's modified Eagle's medium (DMEM) from GIBCO BRL (Grand Island, N.Y.), Whatman GF/B glass fiber filters from Fischer Scientific (Pittsburg, Pa.), fetal calf serum (FCS) and fetal bovine serum (FBS) from HyClone Laboratories (Logan, Utah) and Budget-Solve scintillation fluid from RPI Corp. (Mount Prospect, Ill.).
Synthesis of compounds. All compounds were synthesized from various intermediates 2a-c prepared using our published procedure (Singer et al., 1998) and as shown in Schemes 2-7 of FIGS. 1-6, starting with the commercially available 5-cyano-dimethoxyresorcinol 1 (Scheme 1, FIG. 1). Compounds listed in Table 1a-c were synthesized from the acid 7 using standard procedures for the preparation of amides. The reverse amides (O-2589, O-2590, O-2619 and O-2620) were synthesized from 5 by conversion to the amine via the azide, followed by condensation with the appropriate acids using either the acid chloride or the carbodiimide (EDCI/DMAP) procedures. All the N-alkylated compounds listed in Table 2a-c were synthesized from 5 by protection of the phenol as the TBDMS derivative, which was treated with the appropriate amine in the presence of NaH/DMF, to give the target compounds. The C-alkylated imidazole derivative (O-2737) was synthesized from 7 by conversion of the acid group to the aldehyde, followed by condensation with glyoxal/NH₃ to form the 2-imidazole derivative (Dhanak et al., 2001). The phenolic esters listed in Table 3a-c were synthesized from 6 using a published procedure (Razdan et al., 1976). The various acids used in their preparation were prepared according to literature procedures (Blicke et al., 1941; Cruickshank and Sheehan, 1961; Razdan et al., 1976). The quartenary compounds were synthesized by the treatment of the amines with CH₃I in ether. The compound listed in Table 4 was synthesized from the amide O-2372 (see Table 1a) and diisopropylaminobutyric acid. HCl using the EDCI/DMAP procedure and the free base thus obtained was converted to its hydrochloride. All compounds showed appropriate ¹H NMR profiles (Jeol Eclipse 300 MHZ; Jeol USA, Inc., Peabody, Mass.) and were characterized on the basis of their ¹H NMR profiles, TLC, and elemental analyses. Detailed synthesis Schemes 1-6 for the compounds are given in FIGS. 1-6, respectively.

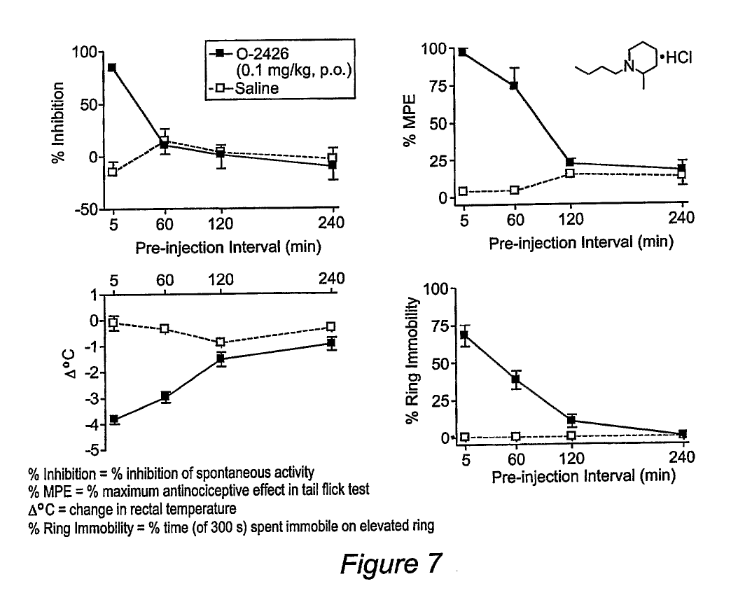

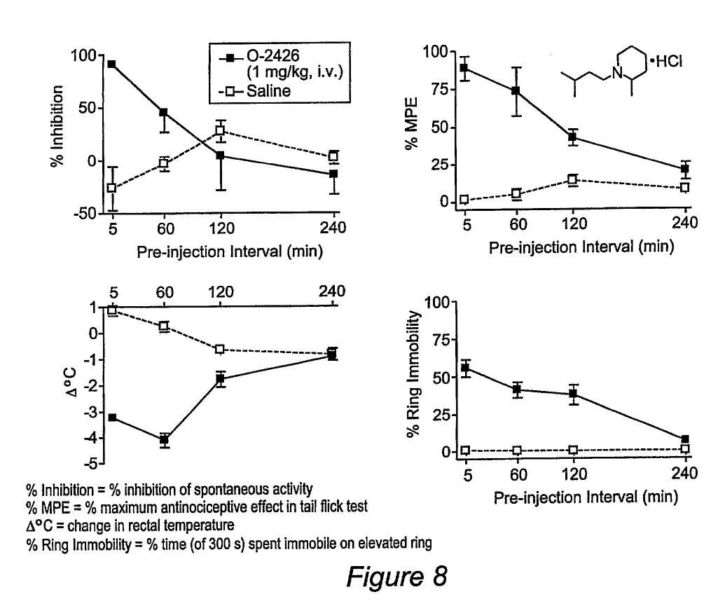

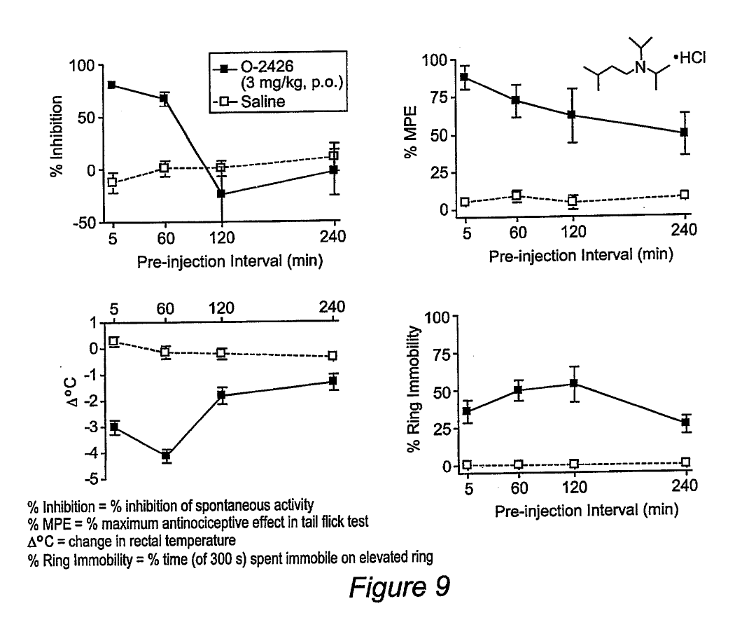

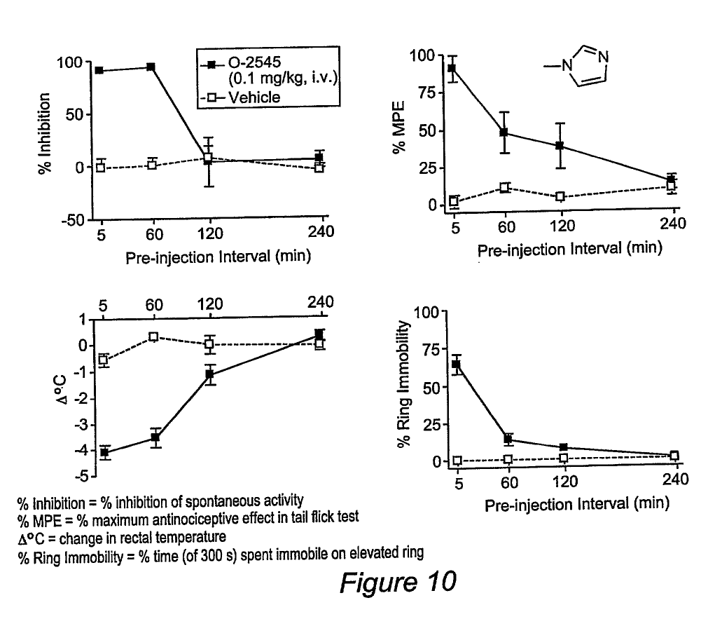

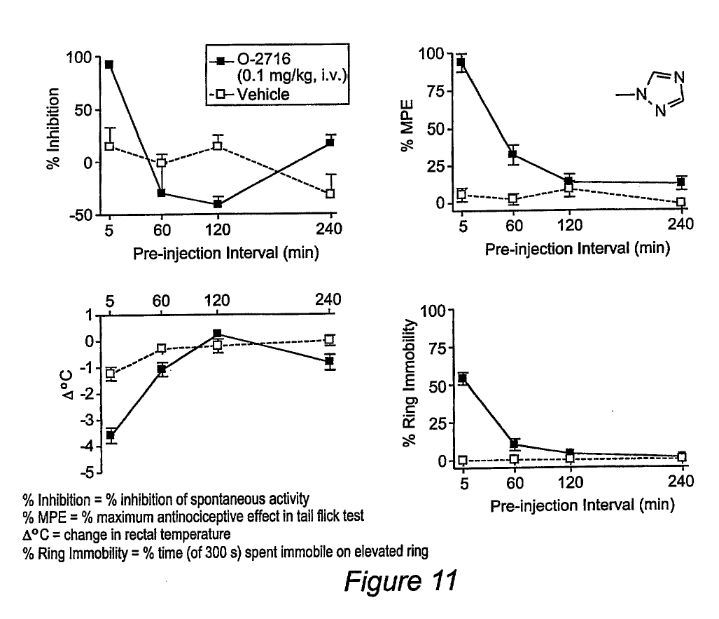

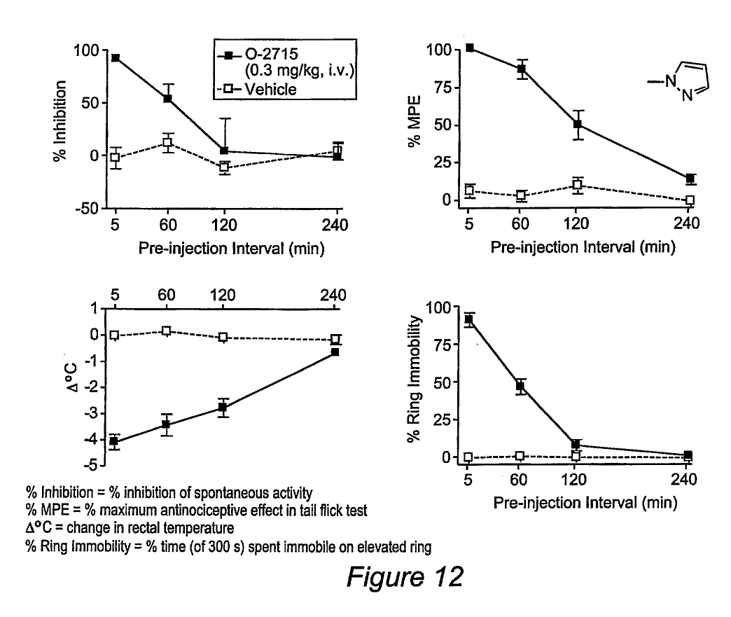

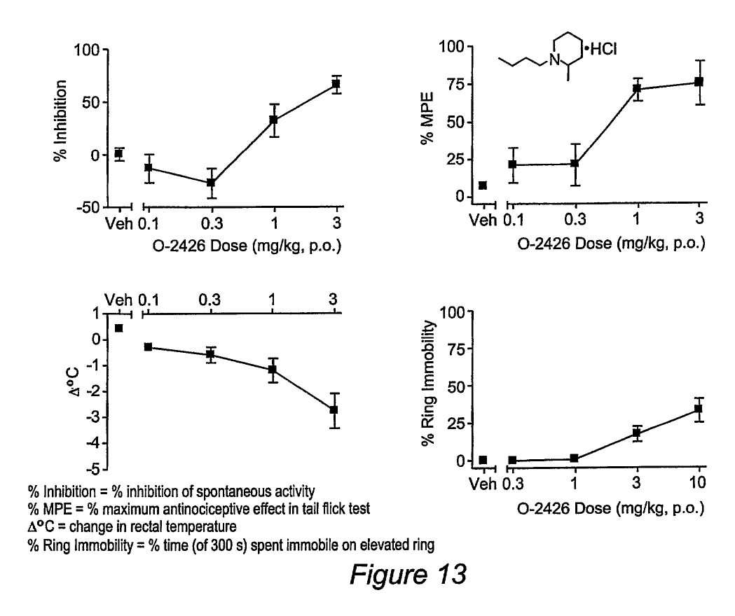

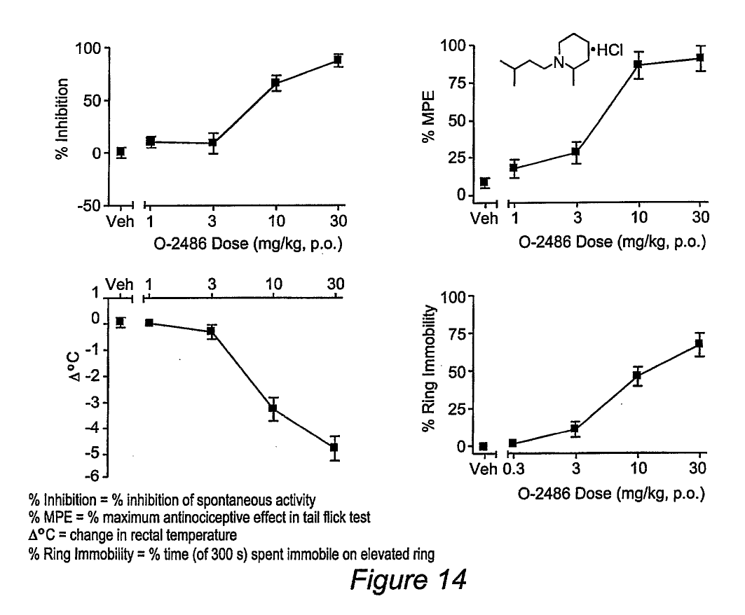

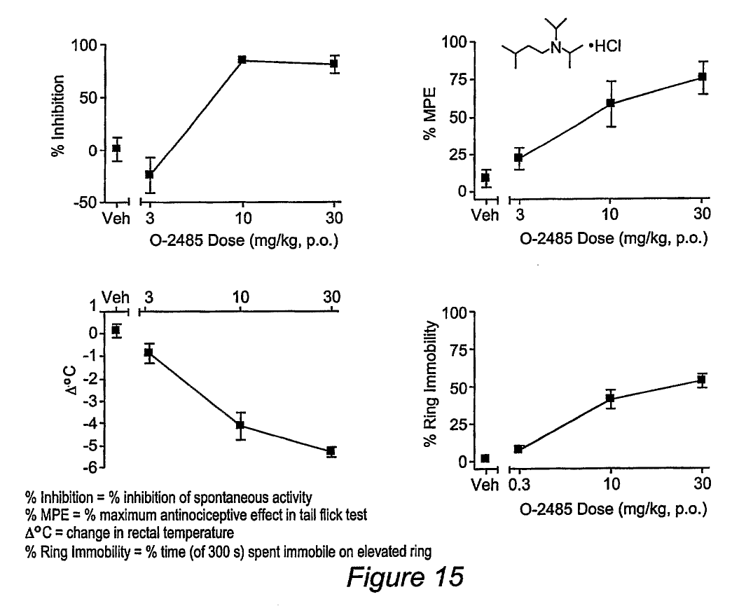

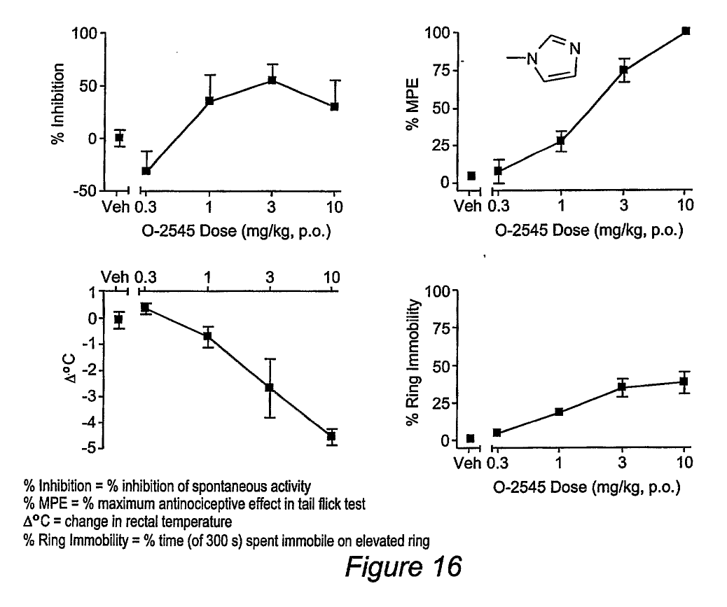

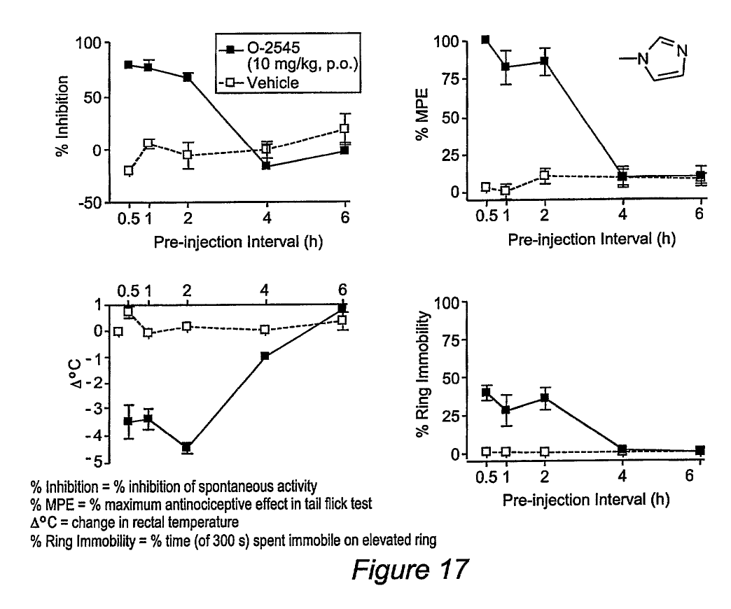

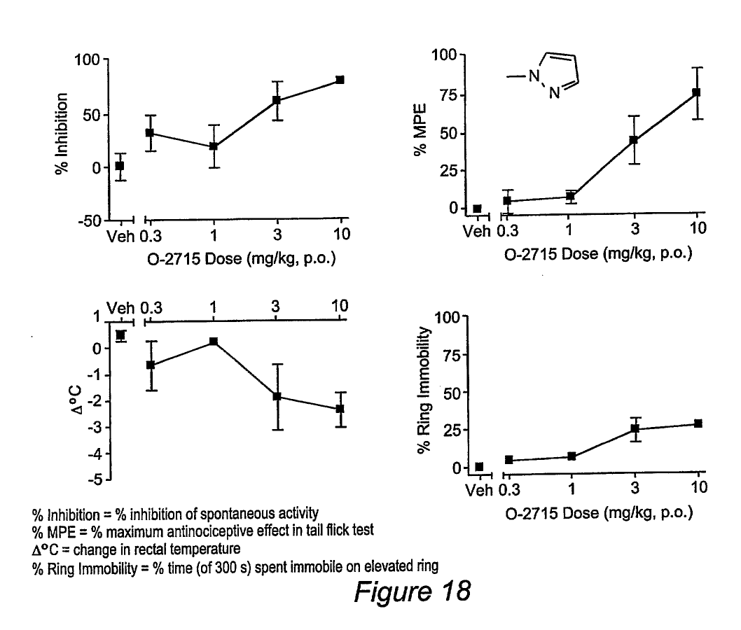

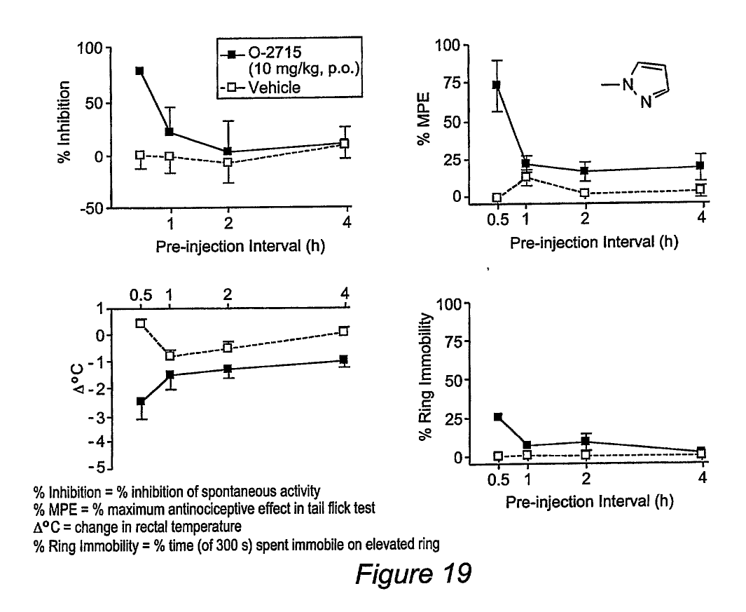

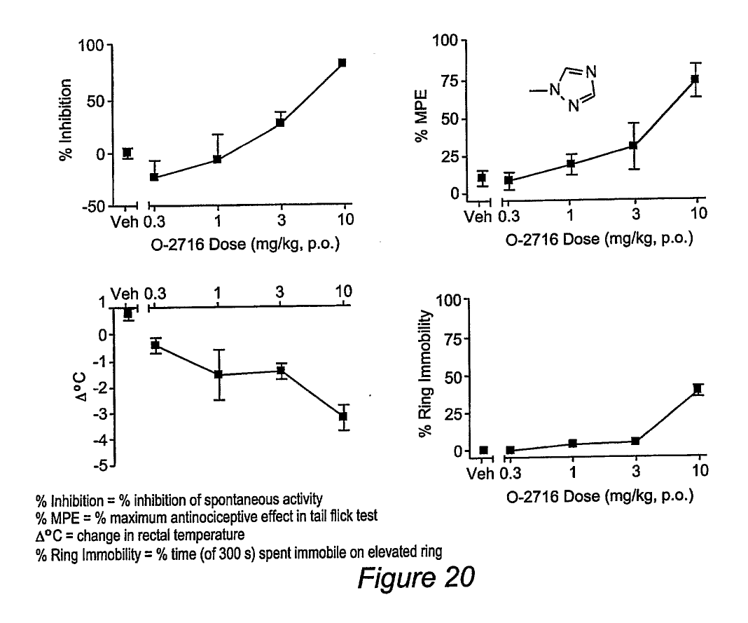

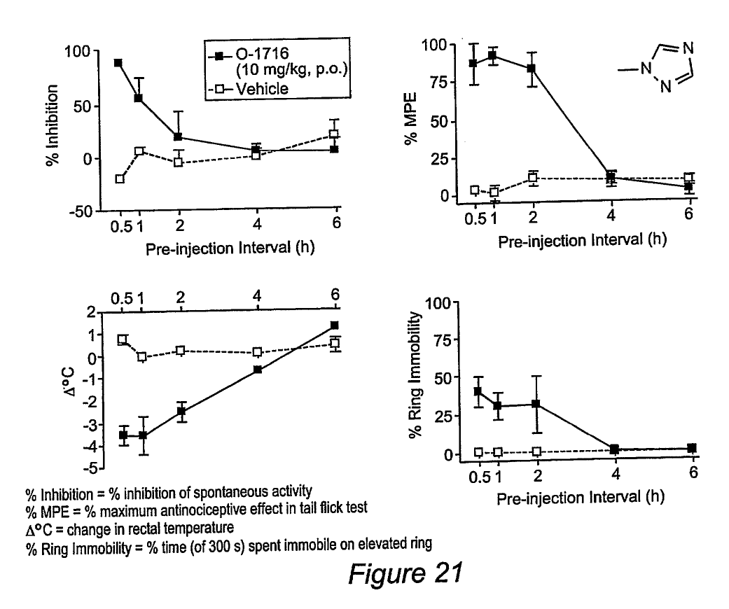

For Tables 1-4, CB₁ and CB₂ receptor binding was carried out in CHO (Chinese hamster ovary) and HEK (human embryonic kidney) cells, respectively, with the exception of the THC and CP 55,940 CB₁ binding, which were performed in rat brain membranes. GTPγS binding was carried out in CHO cells and maximal binding was expressed as a percentage of stimulation produced by CP 55,940. For the in vivo studies, the drugs were dissolved in either saline or emulphor:ethanol:saline (E:E:S) as indicated. The ED50 values are provided as μmoles/kg for reducing spontaneous activity (S.A), producing antinociception in the tail-flick procedure (T.F., lowering rectal temperature (R.T.) And producing relative immobility (R.I.) In mice. Solubility was determined by dissolving 1 mg of analog in 1 ml of water and by visual observation. “N.D.” means “not determined”. The results are presented graphically in FIGS. 7 through 21.

TABLE 1A

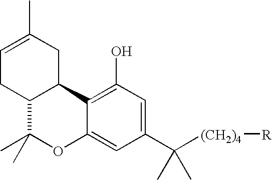

Carboxamido Pentyl Side Chain Analogs
n

| O# | Name | Structure or R group |
|---|---|---|
| | Δ⁹-THC | 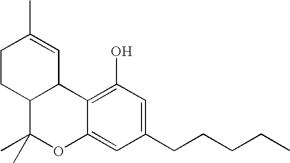 |
| | CP 55,940 | 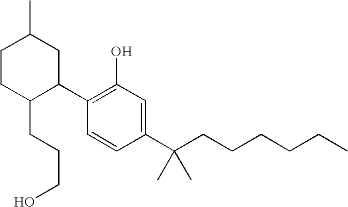 |
| O-2352 | Carboxamido | —CONH₂ |
| O-2490 | N-(Methyl)carboxamido | —CONHCH₃ |
| O-2544 | N-(Morpholin-1-yl)-carboxamido | 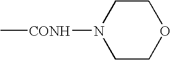 |
| O-2489 | N-(Homo-piperidin-1-yl)-carboxamido | 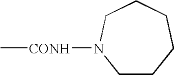 |
| O-2543 | N-(Pyrrolidin-yl)-carboxamido | 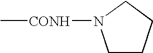 |
| O-2372 | Morpholino-carboxamido | 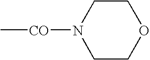 |
| O-2373 | Piperidino-carboxamido | 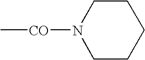 |
| O-2381 | Methylepiperazino-carboxamido | 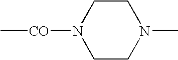 |
| O-2399 | Pyrrolidino-carboxamido | 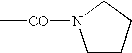 |
| O-2421 | Homopiperidino-carboxamido | 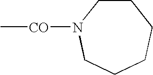 |
| O-2589 | 2,4-Dimethyl-thiazole-5-carboxamide | 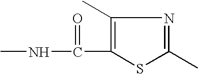 |
| O-2590 | 5-Methyl-2-phenyl-oxazol-4-yl-acetamide | 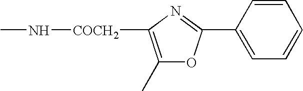 |
| O-2619 | Morpholino-1-carboxylic acid amide | 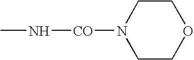 |
| O-2620 | Di(morpholino-1-carboxylic acid) amide | 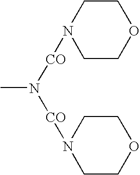 |

TABLE 1B
Carboxamido Pentyl Side Chain Analogs

| | | | CB₁ | CB₁ |
|---|---|---|---|---|
| O# or Name | CB₁ Ki | CB₂ Ki | EC50 | % CP Stim |
| Δ⁹-THC | 41.0 | 49.1 ± 5.11 | | |
| CP 55,940 | 0.9 ± 0.2 | | | 200 |
| O-2352 | 13.1 ± 0.61 | 0.84 ± 0.05 | 42 ± 4.0 | 95 ± 0.30 |
| O-2490 | 18.7 ± 0.58 | 3.16 ± 0.75 | 64.2 ± 10.6 | 92.7 ± 7.81 |
| O-2544 | 5.97 ± 0.65 | 11.4 ± 0.91 | 39.7 ± 11.4 | 116 ± 3.71 |
| O-2489 | 15.8 ± 0.44 | 35.3 ± 4.48 | 86.6 ± 17.4 | 112 ± 2.32 |
| O-2543 | 23.3 ± 3.40 | 10.8 ± 0.08 | 81.13 ± 40.02 | 71.3 ± 4.23 |
| O-2372 | 1.30 ± 0.12 | 0.57 ± 0.04 | 5.88 ± 0.42 | 120 ± 1.11 |
| O-2373 | 0.96 ± 0.11 | 0.96 ± 0.01 | 13.5 ± 1.81 | 119 ± 2.05 |
| O-2381 | 112 ± 14 | 389 ± 46 | ND | ND |
| O-2399 | 2.85 ± 0.52 | 2.86 ± 0.68 | 275 ± 87.9 | 117 ± 4.90 |
| O-2421 | 4.24 ± 1.01 | 3.45 ± 0.58 | 23.2 ± 2.06 | 81.8 ± 8.96 |
| O-2589 | 244 ± 28.5 | 38.4 ± 7.62 | ND | ND |
| O-2590 | 890 ± 161 | 169 ± 39.1 | ND | ND |
| O-2619 | 18.6 ± 3.94 | 2.26 ± 0.38 | 86 ± 3.10 | 116 ± 7.94 |
| O-2620 | 3020 ± 579 | 772 ± 60.5 | ND | ND |

TABLE 1C
Carboxamido Pentyl Side Chain Analogs

| O# or | S.A. | T.F. | R.T. | R.I | | |
|---|---|---|---|---|---|---|
| Name | (μmole/kg) | (μmole/kg) | (μmole/kg) | (μmole/kg) | Solubility | Clogp |
| Δ⁹-THC | 2.23 | 2.77 | 2.23 | | No | 7.238 |
| CP 55,940 | 0.11 | 2.77 | 0.93 | 0.92 | No | 5.819 |
| O-2352 | 0.16 | 0.84 | 1.72 | 0.93 | No | 5.699 |
| O-2490 | 0.49 | 1.37 | >1 | >1 | No | 5.735 |
| O-2544 | 0.17 | 0.48 | 0.25 | 0.63 | No | 5.869 |
| O-2489 | 2.07 | 2.53 | 8.50 | 20.49 | No | 7.153 |
| O-2543 | 0.71 | 0.75 | 8.19 | 25.83 | No | 6.035 |
| O-2372 | 0.01 | 0.01 | 0.07 | 0.03 | No | 6.000 |
| O-2373 | 0.02 | 0.03 | 0.03 | 0.04 | No | 6.794 |
| O-2381 | 21.43 | 34.03 | 51.05 | 182.56 | No | 6.561 |
| O-2399 | 0.03 | 0.10 | 0.23 | 0.15 | No | 6.235 |
| O-2421 | 0.15 | 0.16 | 0.44 | 0.86 | No | 7.353 |
| O-2589 | ND | ND | ND | ND | No | 7.437 |
| O-2590 | ND | ND | ND | ND | No | 7.499 |
| O-2619 | ND | ND | ND | ND | No | 6.160 |
| O-2620 | ND | ND | ND | ND | ND | 7.925 |

TABLE 2a

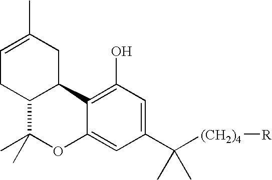

Imidazole, Pyrozole, and Triazole Pentyl Side Chain Analogs

| | Name | |
|---|---|---|
| | Δ⁹-THC | |
| O# | CP 55,940 | R |
| O-2545 | Imidazol-1-yl | 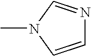 |
| O-2545 | Imidazol-1-yl | 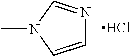 |
| O-2651 | 2-Methyl-imidazol-1-y1 | 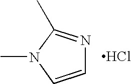 |
| O-2715 | Pyrazol-1-yl | 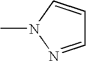 |
| O-2716 | 1,2,4-Triazol-1-yl | 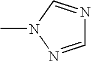 |
| O-2737 | 1H-Imidazol-2-yl | 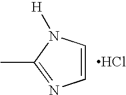 |
| O-2737 | 1H-Imidazol-2-yl | 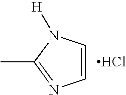 |

TABLE 2b
Imidazole, Pyrozole, and Triazole Pantyl Side Chain Analogs

| | | | CB₁ | | |
|---|---|---|---|---|---|
| O# or Name | CB₁ Ki (nM) | CB₂ Ki (nM) | EC (50) | CB₁ % CP Stim | Vehicle |
| Δ⁹-THC | 41.0 | 49.1 ± 5.11 | | | |
| CP 55,940 | 0.9 ± 0.2 | | | 100 | |
| O-2545 | 1.34 ± 0.17 | 0.12 ± 0.003 | 29.3 ± 3.27 | 107 ± 6.19 | E:E:S |
| O-2545 | 1.47 ± 0.22 | 0.32 ± 0.02 | 4.57 ± 0.72 | 84 ± 5.01 | Saline |
| O-2651 | 13.9 ± 0.83 | 1.22 ± 0.28 | 82.9 ± 45.7 | 33.4 ± 3.41 | Saline |
| O-2715 | 1.94 ± 0.29 | 1.52 ± 0.31 | 4.03 ± 0.28 | 93.0 ± 1.75 | E:E:S |
| O-2716 | 3.43 ± 0.16 | 0.92 ± 0.07 | 36.8 ± 4.23 | 93.1 ± 1.65 | E:E:S |
| O-2737 | 54.0 ± 4.91 | 14.8 ± 1.14 | ND | ND | Saline |
| O-2737 | 54.9 ± 4.91 | 14.8 ± 1.14 | ND | ND | E:E:S |

TABLE 2c
Imidazole, Pyrozole, and Triazole Pantyl Side Chain Analogs

| O# or | S.A | T.F. | R.T. | R.I. | | |
|---|---|---|---|---|---|---|
| Name | (μmole/kg) | (μmole/kg) | (μmole/kg) | (μmole/kg) | Solubility | Clogp |
| Δ⁹-THC | 2.23 | 2.77 | 2.23 | | | 7.238 |
| CP 55,940 | 0.11 | 2.77 | 0.93 | 0.92 | | 5.819 |
| O-2545 | 0.01 | 0.07 | 0.04 | 0.13 | No | 5.860 |
| O-2545 | 0.09 | 0.16 | 0.29 | 0.14 | Yes | 5.860 |
| O-2651 | 1.46 | 1.75 | 3.58 | 4.29 | Yes | 7.069 |
| O-2715 | 0.004 | 0.06 | 0.14 | 0.07 | No | 7.070 |
| O-2716 | 0.03 | 0.09 | 0.15 | 0.20 | No | 6.070 |
| O-2737 | >2.2 | >2.2 | >2.2 | >2.2 | Yes | 6.646 |
| O-2737 | >10 | >10 | >10 | >10 | Yes | 6.646 |

TABLE 3a

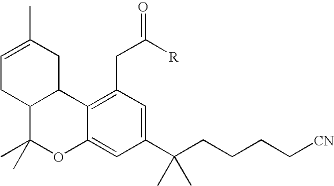

Pharmacological Activity of Phenolic Esters

| | Name | |
|---|---|---|
| | Δ⁹-THC | |
| O# | CP 55,940 | R |
| O-1057 | morpholinobutyryloxy | 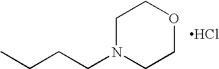 |
| O-2365 | 1-(4-N-piperidinobutyryloxy) | 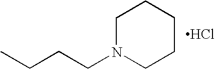 |
| O-2374 | 1-(2-methyl-4-N-piperidinobutyryloxy) | 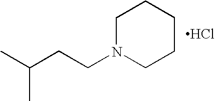 |
| O-2426 | 1-(4-N-2-methylpiperidinobutyryloxy) | 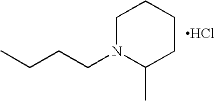 |
| O-2486 | 1-2-methyl-(4-N-2′-methylpiperidino-butyryloxy) | 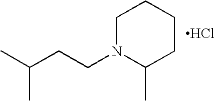 |
| O-2383 | 1-(4-N(4′-methylpiperazino)butyryloxy | 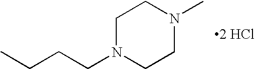 |
| O-2427 | 1-2-methyl-4-N(4′-methyl-piperazino)butyryloxy | 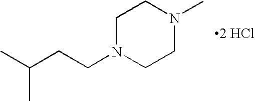 |
| O-2484 | 1-(4-N,N-dimethylaminobutyryloxy) | 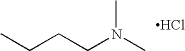 |
| O-2487 | 1-(2-methyl-4,N,N-dimethylaminobutyryloxy) | 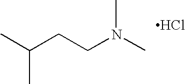 |
| O-2548 | 1-(4-N,N,N-trimethylammoniumbutyryloxy) | 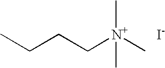 |
| O-2650 | 1-(2-methyl-4-N,N,N-trimethylammoniumbutyryloxy | 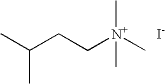 |
| O-2382 | 1-(4-N,N-diisopropylaminobutyryloxy) | 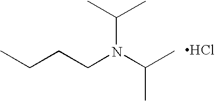 |
| O-2485 | 1-(2-methyl-4-N,N-diisopropylaminobutyryloxy) | 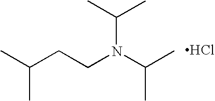 |

TABLE 3b
Pharmacological Activity of Phenolic Esters

| O# or Name | CB₁ Ki (nM) | CB₂ Ki (nM) | CB₁ EC (50) | CB₁ % CP Stim | Vehicle |
|---|---|---|---|---|---|
| Δ⁹-THC | 41.0 | 49.1 ± 5.11 | | | |
| CP 55,940 | 0.9 ± 0.2 | | | 100 | |
| O-1057 | 15.3 ± 5 | ND | ND | ND | E:E:S |
| O-2365 | 2.73 ± 0.83 | 0.20 ± 0.07 | 8.43 ± 2.32 | 93 ± 0.68 | Saline |
| O-2374 | 4.62 ± 2.20 | 1.10 ± 0.10 | 16.5 ± 6.97 | 80.9 ± 3.73 | Saline |
| O-2426 | 2.63 ± 0.55 | 0.70 ± 0.09 | 22.1 ± 7.79 | 77.0 ± 5.87 | Saline |
| O-2486 | 11.3 ± 1.40 | 0.34 ± 0.04 | 36.8 ± 5.98 | 81.3 ± 17.9 | Saline |
| O-2383 | 2.01 ± 0.14 | 0.78 ± 0.13 | 15.9 ± 3.11 | 82.0 ± 5.84 | Saline |
| O-2427 | 6.71 ± 1.80 | 1.56 ± 0.47 | 15.6 ± 7.76 | 97.9 ± 10.4 | Saline |
| O-2484 | 2.61 ± 0.83 | 0.23 ± 0.03 | 16.9 ± 1.74 | 126 ± 11.3 | Saline |
| O-2487 | 5.89 ± 0.79 | 0.44 ± 0.07 | 16.1 ± 6.03 | 119 ± 12.2 | E:E:S |
| O-2548 | 14.7 ± 4.00 | 0.35 ± 0.03 | 60.2 ± 13.2 | 112 ± 1.18 | E:E:S |
| O-2650 | 45.4 ± 3.79 | 7.4 ± 0.24 | 138 ± 35.8 | 113 ± 4.65 | Saline |
| O-2382 | 5.59 ± 1.90 | 1.98 ± 0.08 | 17.9 ± 1.03 | 77.6 ± 5.58 | Saline |
| O-2485 | 81.7 ± 11.3 | 1.99 ± 0.24 | ND | ND | Saline |

TABLE 3c
Pharmacological Activity of Phenolic Esters

| O# or | S.A | T.F. | R.T. | R.I. | | |
|---|---|---|---|---|---|---|
| Name | (μmole/kg) | (μmole/kg) | (μmole/kg) | (μmole/kg) | Soluble | Clogp |
| Δ⁹-THC | 2.23 | 2.77 | 2.23 | | | 7.238 |
| CP 55,940 | 0.11 | 0.23 | 0.93 | 0.92 | | 5.819 |
| O-1057 | 0.03 | 0.03 | 0.10 | | Yes | 6.934 |
| O-2365 | 0.02 | 0.07 | 0.03 | 0.10 | Yes | 6.419 |
| O-2374 | 0.18 | 0.41 | 0.72 | 0.57 | Yes | 8.457 |
| O-2426 | 0.05 | 0.04 | 0.10 | 0.11 | Yes | 8.523 |
| O-2486 | 0.07 | 0.33 | 2.76 | 0.94 | Yes | 8.832 |
| O-2383 | 0.03 | 0.04 | 0.09 | 0.09 | Yes | 6.031 |
| O-2427 | 0.23 | 0.17 | 0.40 | 0.22 | Yes | 6.314 |
| O-2484 | 0.07 | 0.09 | 0.35 | 0.16 | Yes | 6.988 |
| O-2487 | 0.40 | 0.26 | 0.40 | 0.59 | Yes | 7.207 |
| O-2548 | 0.02 | 0.05 | 0.09 | 0.08 | Yes | 7.845 |
| O-2650 | 0.0002 | 1.99 | 3.82 | 14.81 | Yes | 8.154 |
| O-2382 | 0.04 | 0.05 | 0.85 | 0.06 | Yes | 8.989 |
| O-2485 | 0.45 | 1.00 | 1.90 | 1.92 | Yes | 8.973 |

TABLE 4a

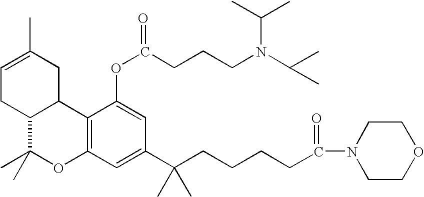

Diisopropylaminobutyrate of Δ⁸-THC-3-(1,1-dimethyl-6-
morpholin-4-yl-6-oxo-hexyl)

| | CB₁ Ki | CB₂ Ki | CB₁ | CB₁ |
|---|---|---|---|---|
| O# | (nM) | (nM) | EC(50) | %CP Stim |
| O-2694 | 3.7 ± 0.43 | 2.77 ± 0.44 | 28.3 ± 3.47 | 120 ± 8.04 |

TABLE 4b
Diisopropylaminobutyrate of Δ⁸-THC-3-(1,1-dimethyl-
6-morpholin-4-yl-6-oxo-hexyl)

| | S.A | | R.T. | R.I. | | |
|---|---|---|---|---|---|---|
| | (μmole/ | T.F. | (μmole/ | (μmole/ | | |
| O# | kg) | (μmole/kg) | kg) | kg) | Clogp | Soluble |
| O-2694 | 0.045 | 0.035 | 0.12 | 0.11 | 8.245 | Yes |

cLogP calculations. Absolute solubility determinations were not performed. Rather, cLogP calculations were performed using the CS ChemDraw Ultra software (Cambridge Soft Corporation, Cambridge, Mass.).
Receptor Binding. HEK-293 cells stably expressing the human CB₁ receptor were cultured in DMEM with 10% FBS and Chinese Hamster Ovary (CHO) cells stably expressing the human CB₂ receptor were cultured in DMEM with 10% FCS. Cells were harvested by replacement of the media with cold phosphate-buffered saline containing 1 mM EDTA followed by centrifugation at 1000×g for 5 min at 4° C. The pellet was resuspended in 50 mM Tris-HCl containing 320 mM sucrose, 2 mM EDTA and 5 mM MgCl2 (pH 7.4) (centrifugation buffer), then centrifuged at 1000×g for 10 min at 4° C., and the resulting supernatant was saved. This process was repeated twice. The supernatant fractions were combined and centrifuged at 40,000×g for 30 min at 4° C. The resulting P2 pellet was resuspended in assay buffer (50 mM Tris-HCl (pH 7.4), 3 mM MgCl₂, 0.2 mM EGTA, and 100 mM NaCl) and protein was determined by the method of Bradford ENRfu(Bradford 1976). Membranes were stored at −80° C. until use. Membrane homogenates (50 μg) were incubated with 0.5 nM [³H]CP55,940 in the presence of varying concentrations (1 nM-10 μM) of test compounds in assay buffer with BSA (5 mg/ml) in a 0.5 ml total volume. Non-specific binding was measured in the presence of 1 μM CP55,940. The assay was incubated at 30° C. for 1 hr and terminated by addition of ice cold 50 mM Tris-HCl plus BSA (1 mg/ml) (pH 7.4) followed by filtration under vacuum through Whatman GF/B glass fiber filters with 3 washes with cold Tris buffer. Bound radioactivity was determined by liquid scintillation spectrophotometry at 50% efficiency after extraction by shaking samples for 30-60 min with Budget-Solve scintillation fluid. Data are reported as the mean±SEM of three experiments, each performed in triplicate. Kᵢ values were calculated from displacement data using EBDA (Equilibrium Binding Data Analysis; BIOSOFT, Milltown, N.J.).
[³⁵S]GTPγS Binding Assays. Concentration-effect curves were generated by incubating membranes (10 μg prepared from CB₁ expressing cells as described above) in assay buffer containing BSA (1 mg/ml) with various concentrations of test compounds in the presence of 20 μM GDP and 0.1 nM [³⁵S]GTPγS in a 1 ml total volume. [³⁵S]GTPγS binding stimulated by 2 μM CP55,940 was used as an internal standard in each assay. Basal binding was assessed in the absence of agonist, and nonspecific binding was measured in the presence of 10 μM GTPγS. The reaction was incubated for 90 min at 30° C. and terminated by filtration under vacuum through Whatman GF/B glass fiber filters with 3 washes with cold (4° C.) Tris buffer (50 mM Tris-HCl, pH 7.4). Bound radioactivity was determined by liquid scintillation spectrophotometry at 95% efficiency after extraction overnight in ScintiSafe Econo 1 scintillation fluid. Data are reported as mean±SEM of three experiments, each performed in triplicate. Nonlinear regression analysis was conducted by iterative fitting using JMP (SAS for Macintosh). Net-stimulated [³⁵S]GTPγS binding is defined as [³⁵S]GTPγS binding in the presence of drug minus basal and percent stimulation is expressed as (net stimulated [³⁵S]GTPγS binding/basal)×100%.
Behavioral Evaluations. All animals were allowed to acclimate to the observation room overnight. Behavioral effects were assessed in the tetrad model in order to measure potency in producing antinociception, catalepsy, hypothermia, and hypomobility. The baselines for tail-flick latency (2-4 seconds) and rectal temperature were determined prior to i.v. injections. Baseline rectal temperatures were measured using a telethermometer and a thermometer probe inserted to 25 mm (Yellow Springs Instrument Co., Yellow Springs, Ohio). The mice were then given either an i.v. i.c.v. injection of the analog. The mice were placed in individual photocell activity chambers 5 min later. Spontaneous activity was monitored for 10 min in a Digiscan Animal Activity Monitor (Omnitech Electronice, Inc., Columbus, Ohio) as measured by the number of interruptions of 16 photocell beams per chamber. The total number of beam interruptions during the 10-min period was determined and presented as total counts. The mice were then assessed at 20 min following the i.v. injection for antinociception using the tail-flick reaction time to a heat stimulus. A 10-sec maximum latency was used in order to avoid tail injury. The results are presented as % MPE and are calculated as follows:

% MPE=[(test latency-control latency)/(10 sec−control latency)]×100

Rectal temperature was measured 30 min after the i.v. injection. The change in rectal temperature (AOC) following analog administration was calculated for each animal. Relative immobility (catalepsy) was measured 40 min after the i.v. injection by the ring-immobility test. Mice were placed on a ring 5.5 cm in diameter attached to a stand at a height of 16 cm. The amount of time the mice spent motionless on the ring during the 5-minute procedure was measured, with the criteria of immobility being defined as the absence of all voluntary movements, including whisker movement, but excluding respiration. The percent immobility was calculated as: % immobility=[time immobile (sec)]/[length of session (sec)]×100. Mice that fell from the ring or actively jumped were allowed five attempts. After the fifth escape these mice were removed from the ring and not included in the calculations. Data was collected from 6-12 mice for each condition tested.

All studies were carried out in accordance with the Declaration of Helsinki and Guide for the Care and Use of Laboratory Animals as adopted and promulgated by the U.S. National Institutes of Health.

Data analysis. Maximal possible effects (100%) served as estimates for the maximal effects for effects on spontaneous activity and tail-flick response. For catalepsy, a maximal possible effect was considered to be 60%, and −6° C. was used as maximal change in rectal temperature based upon data from numerous previous studies with classical cannabinoid compounds (Martin et al., 1991; Compton et al., 1993). ED₅₀ was defined as the dose at which half maximal effect occurred. For compounds that were active in one or more test, ED₅₀s were calculated separately using least-squares linear regression on the linear part of the dose-effect curve for each measure in the mouse tetrad, plotted against log₁₀ transformation of the dose.

### Results

Carboxamido Pentyl Side Chain Analogs. There is ample evidence that a wide range of structural alterations at the terminal carbon of a dimethypentyl chain can be made without diminishing pharmacological activity. For example, addition of a cyano moiety at this position greatly enhances CB₁ receptor affinity and pharmacological potency (Martin et al., 1999). Therefore, a series of carboxamido derivatives were prepared as shown in Table 1. Most of the analogs exhibited high affinity for both CB₁ and CB₂ receptors, were effective in stimulating [³⁵S]GTPγS binding and produced in vivo cannabinoid effects. The unsubstituted carboxamido (O-2352) exhibited excellent CB₁ receptor affinity and even higher CB₂ receptor affinity. Agonist-stimulated [³⁵S]GTPγS binding indicated it was a potent and fully efficacious agonist. hi the in vivo mouse model, it was slightly more potent than Δ⁹-THC following i.v. administration. Moreover, its lipophilicity was calculated to be 35-fold less than of Δ⁹-THC. CB₁ receptor affinity, efficacy ([³⁵S]GTPγS binding) and pharmacological potency were retained with carboxamido substitutions containing methyl (O-2490), morpholinyl (O-2544), homo-piperidinyl (O-2489) or pyrrolidinyl (O-2543) moieties, although there were several notable differences between these compounds. The CB2 receptor selectivity was greatly diminished with all substitutions and was actually reversed with the morpholino (O-2372) and piperdino (O-2373) analogs. Interestingly, the homo-piperidinyl (O-2489) and pyrrolidinyl (O-2543) analogs were 4-25 fold less potent in producing hypothermia and catalepsy than in producing hypoactivity and analgesia. In addition, 0-2490 failed to produce maximal effects in hypothermia and catalepsy assays at a dose (1 mg/kg) that produced maximal effects in the other two measures. A separation in pharmacological potencies of this magnitude is unusual. The calculated lipophilicity was similar for all of the aryl hydrazines with the exception of the homo-piperidinyl (O-2489) analog that was found to be 28 times more lipid soluble than the unsubstituted carboxamido analog (O-2352).

Five carboxamido analogs were prepared with the morpholino (O-2372), piperidino (O-2373), pyrrolidino (O-2399), and homo-piperidino (O-2421) having high CB₁ and CB₂ receptor affinity, high efficacy in [³⁵S]GTPγS binding, and very high in vivo potency in all four pharmacological measures. The most active compounds (O-2372) and (O-2373) were 100-200 times more potent than Δ⁹-THC after i.v, administration. It is noteworthy that a simple change from a piperidine to a piperazine (O-2381) decreased CB₁ receptor affinity and pharmacological potency more than a hundred fold. Most of these compounds were slightly less lipophilic than Δ⁹-THC. Of the remaining compounds in Table 1, only O-2619 demonstrated reasonable affinity for the CB₁ receptor; however, it was not evaluated in vivo.

Compounds O-2352, O-2372, O-2373, O-2399, and O-2544 were converted to hydrochloride salts as confirmed by NMR. However, none of the HCl salts was water-soluble. These analogs were deemed representative of the analogs in Table 1, and therefore no attempts were made to convert the other analogs in this table to HCl salts. Due to lack of water-solubility, all of the analogs in Table 1 were administered to mice using the 1:1:18 vehicle. Imidazol-, Pyrazol-, and Triazol-pentyl Side Chain Analogs. The imidazol substitution (O-2545) on the terminal carbon atom of the side chain resulted in a high affinity CB₁ agonist that was fully efficacious in stimulating [³⁵S]GTPγS binding (Table 2). It exhibited even higher affinity for the CB₂ receptor. The free base of this analog, dissolved in 1:1:18 vehicle, produced the fall spectrum of pharmacological effects in the mouse model and proved to be at least 40-fold more potent than Δ⁹-THC in all measures. The hydrochloride salt of O-2545 was dissolved in saline and when administered to mice produced effects that were equivalent to those of the free base. The calculated lipophilicity (clogP) of this analog was 20 times less than that of Δ⁹-THC. A single methyl substitution at the 2 position of the imidazole (O-2651) reduced affinity for both CB₁ and CB₂ receptors: however, it was still water soluble. This relatively minor structural alteration had a greater impact on efficacy in that it was only a partial CB₁ receptor agonist in stimulating [³⁵S]GTPγS binding. When administered in saline to mice, it was at least 10-fold less potent than the corresponding hydrochloride salt of O-2545. Both pyrazole (O-2715) and triazole (O-2716) analogs had high CB₁ receptor affinity and fully efficacious in stimulating [³⁵S]GTPγS binding. They were also very potent in producing cannabinoid effects in the tetrad model. However, neither of these analogs was water-soluble. Attempts were made to prepare hydrochloride salts, but the salts could not be isolated because both compounds are weak bases. Merely changing the attachment position from the nitrogen on the imidazole (O-2545) to the carbon 2 position on the imidazole ring (O-2737) dramatically reduced CB₁ and CB₂ receptor affinities. As expected based upon their CB₁ receptor affinity, they exhibited only weak in vivo pharmacological activity. Placement of a morphino group at the terminal carbon position resulted in O-3226 that had high affinity for both CB₁ and CB₂ receptors, was water-soluble, and when dissolved in saline was very potent when administered to mice.

Phenolic esters. The phenolic esters in Table 3 represent an extension of earlier work on the morpholinobutyryloxy derivative of Δ⁸-THC (O-1057). As with O-1057, most of these analogs were water soluble upon conversion to hydrochloride salts. Substituting the morpholino with a piperidino (O-2365) or methypiperidino groups (O-2426) slightly increased CB₁ receptor affinity over that of O-1057. Both analogs were effective in stimulating [³⁵S]GTPγS binding and very potent in the mouse behavioral assays. A methyl group was added to the carbon 1 position in the butyryl moiety in an attempt to delay hydrolysis and to increase chemical stability. This addition produced only a slight reduction in CB₁ receptor affinity and pharmacological potency. The greatest difference was seen with lipophilicity which was increased almost 100-fold by the addition of the methyl group to O-2365 but not to O-2426. The N-4′-methylpiperazino analogs (O-2383 and O-2427) exhibited properties similar to the piperidino (O-2365) and N-2′-methyl-4-piperidino (O-2374) analogs. A series of N-alkylaminobutyryloxy analogs (O-2484, O-2487, O-2548, O-2650, O-2382, and O-2485) was prepared. For the most part, all of these analogs had good CB₁ receptor affinity, stimulated [³⁵S]GTPγS binding, and were potent when administered to mice (Table 3). The 2-methyl analogs (O-2650 and O-2485) were somewhat less potent pharmacologically and had lower CB₁ receptor affinity. It is particularly noteworthy that the quaternary analog O-2548 is very potent pharmacologically. Of course, it also has high lipophilicity despite being a quaternary compound.
4-Diisopropylaminobutyrate of Δ⁸-THC-3-(1,1-dimethyl-6-morpholin-4-yl-6-oxo-hexyl. The fact that the diisopropylaminobutyrate of Δ⁸-THC (O-2382 in Table 3) had high receptor affinity and was extremely potent in vivo provided an opportunity to convert one of the water insoluble carboxamido analogs in Table 1 to a water soluble cannabinoid. Therefore, the morpholinocarboxamido (O-2372) was chosen, because it also has high receptor affinity and excellent in vivo potency despite being water-insoluble. The resulting analog, O-2694, was found to be water-soluble, retained high CB₁ and CB₂ receptor affinity, and exhibited high potency and efficacy in stimulating [³⁵S]GTPγS binding (Table 4). It was also highly potent in vivo despite an almost 200-fold increase in lipophilicity as compared to O-2372. However, according to cLogP values, it is only five-fold less lipophilic than O-2382.
Influence of route of administration. The absorption of THC following oral administration is both poor and erratic. It is assumed that the high lipophilicity of THC contributes to some extent to its low absorption. Therefore, the time course of three analogs with varying cLogP values was examined following their oral administration in mice. O-2545 24 times less lipophilic than Δ⁹-THC) was administered at a dose of 10 mg/kg and different groups of mice were tested at 0.5, 1, 2, 4 and 6 hrs. The time course was similar for all four behavioral measures. For example, maximal analgesia in the tail-flick procedure was obtained at 30 min and decreased to only 85±11% (mean±sem) at two his. By four hrs the effects had largely dissipated. O-2716 is 15 fold less lipophilic than Δ⁹-THC. Its oral administration at a dose of 10 mg/kg resulted in a time course very similar to that of O-2545. Maximal effects were observed at 30 min and only a slight decrease in activity was observed at 2 hrs. At four hrs, few differences were observed between vehicle- and O-2716-treated mice. The lipophilicities of O-2715 and Δ⁹THC are almost identical. As predicted, the oral administration of O-2715 (10 mg/kg) produced negligible effects at one hr (21±6% analgesia), and the effects further declined over time.

In order to determine whether these analogs would be active when injected into a specific site, several analogs were dissolved in water and injected i.c.v. The effects of these compounds were compared to that of THC (dissolved in emulphor:ethanol:saline) and to the O-1057, the hydrochloride salt of morpholinobutyryloxy analog of 5′-cyano-THC. The results in Table 5 show that THC was active with similar potencies in all tests, with the exception of weak antinociceptive activity. The morpholinobutyryloxy analog O-1057 was also active in all behavioral tests, albeit with considerable differences in potency between tests. It was considerably more potent in lowering body temperature and producing catalepsy than in decreasing spontaneous activity and producing antinociception. The 1-(4-N(4′-methylpiperazino)-butyryloxy) analog O-2383 demonstrated potency similar to that of O-1057 with the exception of a low ability to reduce body temperature. The remaining three analogs were imidazole derivatives (side chain) that were also quite potent. Of these three compounds, O-2545 was the most potent. There were also some differences in potencies among the four tests for each analog. However, a consistent pattern did not emerge with all of the compounds, although they did appear to be somewhat less potent in producing antinociception in the tail-flick assay.

### Discussion

Extensive structure activity relationship studies began almost immediately after the structure of THC was established (Gaoni and Mechoulam, 1964; Edery et al., 1971; Razdan, 1986). These initial studies firmly established the importance of three structural features of THC: the C9 position, phenolic hydroxyl group, and pentyl side chain. These regions of the molecule continue to be the focus of structural modifications of THC. Elimination or structural modification of the phenolic hydroxyl group renders THC inactive at the CB₁ receptor (Razdan, 1986). Recently, a series of desoxy-THC analogs were found to lack CB₁ receptor affinity but retained their ability to interact with CB₂ receptors (Huffman et al., 2002). The side chain can easily be manipulated to increase agonist potency or to reduce or eliminate agonist efficacy (Martin et al., 1999). In addition, the terminal carbon atom of the side chain can tolerate a wide range of substituents (Martin et al., 1999). Therefore, structural modifications at the phenolic hydroxyl and side chain represent logical sites for expanding the structure-activity relationship of THC and for developing water-soluble ligands.

As we had previously shown (Zitko et al., 1972; Pertwee et al., 2000), the addition of a basic functional group through an ester linkage at the phenolic hydroxyl group provides a means for preparing hydrochloride salts that are water-soluble. It is now evident that hydrochloride salts can be readily prepared when the terminal group in the butyryloxy group is either morpholino, piperidino or piperazino moieties. Since a free phenolic group is essential for interaction with the CB₁ receptor, it would appear that all of the compounds are readily hydrolyzed, since they are highly potent when administered i.v. to mice. In an effort to retard hydrolysis, we incorporated a methyl group on the alpha carbon of several analogs. While we have no direct evidence of the degree of hydrolysis that occurs in the receptor binding assays, it is noteworthy that the CB₁ receptor affinity decreased with the methyl addition, whereas the CB₂ receptor was less affected. These observations are consistent with decreased hydrolysis since a free hydroxyl is essential for CB₁ but not CB₂ receptor binding. It is also evident that an alkyl substituted amino or ammonium group forms a hydrochloride salt that is in turn water-soluble. Alpha methylation in the butyryloxy group had mixed results on CB₁ and CB₂ binding. In the case of the ammoniumbutyryloxy analogs (0-2548 and O-2650, Table 3), binding was increased for both receptor subtypes. However, binding was increased only for the CB₁ receptor with the dimethyaminobutyryloxy (O-2484 and O-2487) and diisopropylaminobutyryloxy (O-2382 and O-2485) analogs. In the latter case, CB₁ receptor binding was increased 15 fold with the alpha methyl addition. There was also considerable diminution in the pharmacological potency of the methyl derivative, again suggestive of decreased hydrolysis.

The second strategy for developing water-soluble derivatives was incorporation of nitrogenous constituents in the side chain that could be converted to hydrochloride salts. Earlier studies had demonstrated that a wide range of small substituents (hydroxy, bromo, cyano, azido, acetanido, etc.) could be incorporated into the terminus of the side chain (ref). Importantly, these studies demonstrated that the nitrogen containing substituents increased CB₁ receptor affinity and pharmacological potency. Therefore, it was not unexpected that the carboxamido (O-2352) and N-methylcarboxamido (O-2490) analogs would have reasonable receptor affinity and be pharmacologically active. The finding that the incorporation of morpholinyl, piperidinyl, homo-piperidinyl and pyrrolidinyl groups on the side chain terminus retains pharmacological activity indicates for the first time that the receptor pharmacophore readily accommodates bulky substituents at this position. Similar findings were observed in the carboxamido series having a morpholino, piperidino, homo-piperidino and a pyrrolidino group with the exception that these analogs exhibited higher receptor affinity and greater pharmacological potency than the corresponding-yl analogs. It is interesting to note that the homo-piperidinyl analog in the carboxamido series (O-2489, Table 1) is considerably less potent than the corresponding homo-piperidino analog (O-2421). In addition, the methylpiperazino (O-2381) was a very weak agonist. These two observations suggest that electrostatic influences are more important than steric effects at this position. On the other hand, the loss in activity of the di(morpholino-l-carboxylic acid) amide (O-2620) is highly likely to be due to steric hinderance. While most of the carboxamido analogs exhibited high affinity and excellent pharmacological potency, it was disappointing that none of the hydrochloride salts was water-soluble. This lack of water solubility is not surprising since it is known that, although the amides can form salts, they are less water-soluble than amine salts which have greater basicity.

In contrast to the carboxamido analogs, the imidazole analogs readily formed hydrochloride salts that were water-soluble, whereas the less basic pyrazole and triazole analogs formed hydrochloride salts which were water-insoluble. The fact that the imidazole-1-yl analog (O-2545, Table 3) had high receptor affinity, efficacy similar to CP 55,940 in stimulating GTPgS binding and high potency in the mouse behavioral assays make it an ideal water-soluble cannabinoid agonist. It is also 24-fold less lipophilic than THC and does not require metabolic conversion to an active constituent. Moreover, it has excellent stability in buffer and plasma and microsomal enzymes as compared to most of the analogs. The pyrazole and triazole analogs also exhibited excellent receptor affinity and high potency in vivo but as stated above, the hydrochloride salts are not water soluble. The dramatic decrease in receptor affinity and pharmacological potency that occurred with the imidazole-2-yl analog (O-2737) demonstrates the importance of electrostatic influences at the side chain terminus.

The question arises as to whether incorporation of both a phenolic ester and a side chain carboxamide (O-2694, Table 4) would provide even greater water solubility. Clearly, this analog retained high affinity and excellent in vivo potency.

In summary, there are numerous effective means of synthesizing water-soluble cannabinoids. The conversion of the phenolic hydroxyl group to an ester that is readily hydrolyzed in vivo is an effective strategy. However, these analogs may be best suited for systemic administration, in contrast to site specific injections, since metabolic activation is required. On the other hand, incorporation of nitrogen-containing rings at the terminal carbon atom of the side chain allows for the preparation of hydrochloride salts that are readily water-soluble. The advantage of these compounds is that they do not require metabolic activation. It is now possible to use these same synthetic strategies to develop water-soluble CB₁ and CB₂ selective agonist and antagonists. The necessity of using solubilizing agents to order to evaluate the pharmacological properties of an agent poses challenges to the investigator. It will be understood by those of skill in the art that there may be a pharmacological interaction between vehicle and test agent. More likely, the vehicle will influence the pharmacokinetics of the test substance that adds an additional challenge when comparing data generated from different labs that utilize various vehicles. Elimination of solubilizing agents as vehicle eliminates possible artifacts arising from these substances.

### REFERENCES

Blicke F F, Wright W B, Jr. and Zienty M F (1941) The action of heat on γ-alkoxybutyryl chlorides. Amer. Chem. Soc. 63:2488-2490.
Compton D R and Martin B R (1990) Pharmacological evaluation of water soluble cannabinoids and related analogs. Life Sci. 46:1575-1585.
Compton D R, Rice K C, De Costa B R, Razdan R K, Melvin L S, Johnson M R and Martin B R (1993) Cannabinoid structure-activity relationships: Correlation of receptor binding and in vivo activities. J. Pharmacol. Exp. Ther. 265:218-226.
Cruickshank P A and Sheehan J C (1961) Intramolecular assistance of decarboxylative acylation. J. Amer. Chem. Soc. 83:2891-2895.
Dhanak D, Christmann L T, Darcy M G, Keenan R M, Knight S D, Lee J, Ridgers L H, Sarau H M, Shah D H, White J R and Zhang L (2001) Discovery of potent and selective phenylalanine derived CCR2 antagonists. Part1. Bioorg. Med. Chem. Lett. 11:1445-1450.
Edery H, Grunfeld Y, Ben-Zvi Z and Mechoulam R (1971) Structural requirements for cannabinoid activity. Ann. N.Y. Acad. Sci. 191:40-53.
ElSohly M A, Harland E C, Benigni D A and Waller C W (1984) Cannabinoids in glaucoma II: The effect of different cannabinoids on intraocular pressure of the rabbit. Curr. Eye Res. 3:841-850.
Gaoni Y and Mechoulam R (1964) Isolation, structure, and partial synthesis of an active constituent of hashish. J. Amer. Chem. Soc. 86:1646-1647.
Harris L S, Carchman R A and Martin B R (1978) Evidence for the existence of specific cannabinoid binding sites. Life Sci. 22:1131-1138.
Huffman J W, Bushell S M, Miller J R, Wiley J L and Martin B R (2002) 1-Methoxy-, 1-deoxy-11-hydroxy- and 11-Hydroxy-1-methoxy-Delta(8)-tetrahydrocannabinols: new selective ligands for the CB(2) receptor. Bioorg Med Chem 10:4119-4129.
Juntunen J, Huuskonen J, Laine K, Niemi R, Taipale H, Nevalainen T, Pate D W and
Jarvinen T (2003a) Anandamide prodrugs. 1. Water-soluble phosphate esters of arachidonylethanolamide and R-methanandamide. Eur J Pharm Sci 19:37-43.
Juntunen J, Vepsalainen J, Niemi R, Laine K and Jarvinen T (2003b) Synthesis, in vitro evaluation, and intraocular pressure effects of water-soluble prodrugs of endocannabinoid noladin ether. J Med Chem 46:5083-5086.
Khanolkar A D, Palmer S L and Makriyannis A (2000) Molecular probes for the cannabinoid receptors. Chem. Phys. Lipids 108:37-52.
Little P J, Kaplan N C and Martin B R (1987) Pharmacological profile of Δ⁹-THC carbamate. Alc. Drug Res. 7:517-523.
Martin B R, Compton D R, Thomas B F, Prescott W R, Little P J, Razdan R K, Johnson M R,
Melvin L S, Mechoulam R and Ward S J (1991) Behavioral, biochemical, and molecular modeling evaluations of cannabinoid analogs. Pharmacol. Biochem. Behav. 40:471-478.
Martin B R, Jefferson R, Winckler R, Wiley J L, Huffman J W, Crocker P J, Saha B and
Razdan R K (1999) Manipulation of the tetrahydrocannabinol side chain delineastes agonists, partial agonists, and antagonists. J. Pharmacol. Exp. Ther. 290:1065-1079.
Nye J S, Voglmaier S, Martenson R E and Snyder S H (1988) Myelin basic protein is an endogenous inhibitor of the high-affinity cannabinoid binding site in brain. J. Neurochem. 50:1170-1178.
Pertwee R G, Gibson T M, Stevenson L A, Ross R A, Banner W K, Saha B, Razdan R K and Martin B R (2000) O-1057, a potent water-soluble cannabinoid receptor agonist with antinociceptive properties. Br. J Pharmacol. 129:1577-1584.
Razdan R K (1986) Structure-activity relationships in cannabinoids. Pharmacol. Rev. 38:75-149.
Razdan R K, Terris B Z, Pars H G, Plotnikoff N P, Dodge P W, Dren A T, Kyncl J and Somani P (1976) Drugs derived from cannabinoids: 2. Basic esters of nitrogen and carbocyclic analogs. J. Med. Chem. 19:454-461.
Singer M, Ryan W J, Saha B, Martin B R and Razdan R K (1998) Potent cyano and carboxamide side-chain analogus of 1′,1′-dimethyl-Δ⁸-tetrahydrocannabinol. J Med. Chem. 41:4400-4407.
Zitko B A, Howes J F, Razdan R K, Dalzell B C, Dalzell H C, Sheehan J C and Pars H G (1972) Water-soluble derivatives of Δ¹-tetrahydrocannabinol. Science 177:442-444.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A water-soluble cannabinoid analog with the general structure 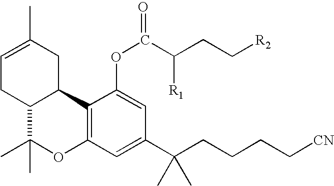
wherein
R₁ is H or a straight-chained, branched or cyclic C₁-C₆ lower alkyl; and
R₂ is
a 5-7 membered heterocyclic ring in which at least one of the member atoms is N; or
NR₃ where R₃ is H₂, H₃⁺, or mono or dialkyl C₁-C₆;
or a salt thereof.

2. The cannabinoid analog of claim 1, wherein said 5-7 membered heterocyclic ring is selected from the group consisting of piperidine, methyl piperidine, methyl piperazine and morpholine.

3. A cannabinoid analog with the general structure 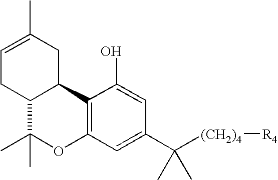
wherein R₄ is
(i) an azole or morpholine ring, or
ii) —CO-R₅ wherein R₅ is NH₂, NHCH₃, or NHR₆, where R₆ is a 5-7 membered heterocyclic ring in which at least one of the member atoms is N; or
wherein R₅ is a 5-7 membered heterocyclic ring in which at least one of the member atoms is N.
or a salt thereof.

4. The cannabinoid analog of claim 3, wherein said azole ring is selected from the group consisting of imidazole, 1H-imidazole, methyl imidazole, pyrazole, and triazole.

5. The cannabinoid analog of claim 3, wherein said cannabinoid analog is a water-soluble salt and said azole ring is selected from the group consisting of imidazole 1H-imidazole, methyl imidazole.

6. (canceled)

7. The cannabinoid analog of claim 3, wherein R₆ is a heterocyclic ring selected from the group consisting of morpholine, homo-piperidine, pyrrolidine, and piperidine.

8. The cannabinoid analog of claim 3, wherein R₅ is a heterocyclic ring selected from the group consisting of morpholine, piperidine, piperizine, pyrrolidine, and homo-piperidine.

9. A water-soluble cannabinoid analog with general structure 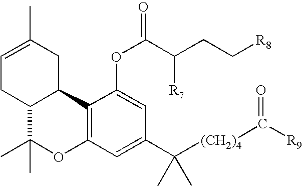
wherein
R₇ is H, CH₃ or a straight-chained, branched or cyclic C₁-C₆ lower alkyl; and
R₈ is
a 5-7 membered heterocyclic ring in which at least one of the member atoms is N; or
NR₃where R₃ is H₂, H₃⁺, or mono or dialkyl C₁-C₆; and
wherein R₉ is NH₂, NHCH₃, or NHR₆, where R₆ is a 5-7 membered heterocyclic ring in which at least one of the member atoms is N;
or
wherein R₉ is a 5-7 membered heterocyclic ring in which at least one of the member atoms is N,
or a salt thereof.

10. The water-soluble cannabinoid analog of claim 9, wherein said analog is

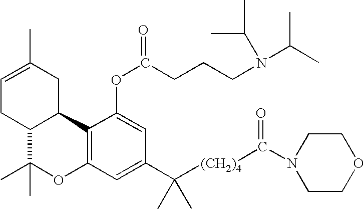

11. A cannabinoid analog with the general structure 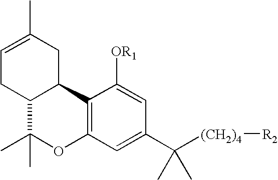
wherein
R₁═H, —COCHR₃—CH₂—CH₂-R₄;
R₂═CN or COR₇;
R₃═H, or a straight-chained, branched or cyclic C₁-C₆ lower alkyl; and
R₄ and R₇ may be the same or different and are
NH₂, NHCH₃, N(R₈)₂, wherein R₈═COR₇;
a 5-7 membered heterocylic ring with at least one N atom, or
NHR₅, where R₅ is a 5-7 membered heterocyclic ring with one N atom.

12. The cannabinoid analog of claim 11, wherein said 5-7 membered heterocyclic ring is

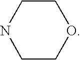

13. A method of treating or alleviating symptoms of a disease or disorder associated with CB1 and CB2 cannabinoid receptors in a patient in need thereof, comprising the step of administering to said patient a compound or salt thereof of a general structural formula 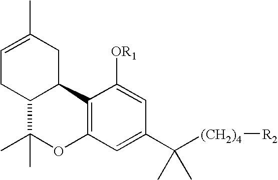
wherein
R₁═H, —COCHR₃—CH₂—CH₂-R₄;
R₂═CN or COR₇;
R₃═H, or a straight-chained, branched or cyclic C₁-C₆ lower alkyl; and
R₄ and R₇ may be the same or different and are
NH₂, NHCH₃, N(R₈)₂, wherein R₈═COR₇;
a 5-7 membered heterocylic ring with at least one N atom, or
NHR₅, where R₅ is a 5-7 membered heterocyclic ring with one N atom.

14. The method of claim 13, wherein said 5-7 membered heterocyclic ring is [chemical structure]
